SEQUENCE LISTING (9 sequences)

SEQ ID NO: 1 - DNA, 20 bp, synthetic construct
  misc_feature 1..20: note = Primer
  source 1..20: mol_type = other DNA
ggagttcaagaaggagtttc

SEQ ID NO: 2 - DNA, 19 bp, synthetic construct
  misc_feature 1..19: note = Primer
  source 1..19: mol_type = other DNA
ccacatacagtgtctggta

SEQ ID NO: 3 - DNA, 20 bp, synthetic construct
  misc_feature 1..20: note = Probe
  misc_feature 1: note = /note="5'FAM"
  misc_feature 20: note = /note="3'BHQ1"
  source 1..20: mol_type = other DNA
ccgcttacgccagaggaact

SEQ ID NO: 4 - DNA, 20 bp, synthetic construct
  misc_feature 1..20: note = Primer
  source 1..20: mol_type = other DNA
agggatttgaatcatgtttg

SEQ ID NO: 5 - DNA, 19 bp, synthetic construct
  misc_feature 1..19: note = Primer
  source 1..19: mol_type = other DNA
ctgctagttctttactggc

SEQ ID NO: 6 - DNA, 23 bp, synthetic construct
  misc_feature 1..23: note = Probe
  misc_feature 1: note = /note="5'HEX"
  misc_feature 23: note = /note="3'BHQ1"
  source 1..23: mol_type = other DNA
tgtagattcaacttgccgctgtc

SEQ ID NO: 7 - DNA, 18 bp, synthetic construct
  misc_feature 1..18: note = Primer
  source 1..18: mol_type = other DNA
ctggaccaaacacaaatg

SEQ ID NO: 8 - DNA, 18 bp, synthetic construct
  misc_feature 1..18: note = Primer
  source 1..18: mol_type = other DNA
atgccttctttcaccttc

SEQ ID NO: 9 - DNA, 20 bp, synthetic construct
  misc_feature 1..20: note = Probe
  misc_feature 1: note = /note="5'Texas red"
  misc_feature 20: note = /note="3'BHQ2"
  source 1..20: mol_type = other DNA
ttgccatccagccactcagt

(19) United States
(12) Patent Application Publication
(10) Pub. No.: US 2025/0282733 A1
(43) Pub. Date: Sep. 11, 2025

(54) NOVEL 1,2,3,4,4A,5,6,7,8,9,10,10A-DODECAHYDROBENZO[G]QUINOLIN-6-OL COMPOUNDS AND USES THEREOF

(71) Applicants: Håkan Wikström, Strömstad (SE); INTEGRATIVE RESEARCH LABORATORIES SWEDEN AB, Göteborg (SE)

(72) Inventors: Håkan Wikström, Strömstad (SE); Per Lindberg, Göteborg (SE); Maria Gullme, Göteborg (SE); Clas Sonesson, Billdal (SE); Sverker Von Unge, Göteborg (SE)

(21) Appl. No.: 18/858,963

(22) PCT Filed: Apr. 24, 2023
(86) PCT No.: PCT/EP2023/060698

§ 371 Date: Oct. 22, 2024

(30) Foreign Application Priority Data
Apr. 25, 2022 (SE) 2250495-5

(51) Int. Cl. C07D 221/08; A61K 31/473

(52) U.S. Cl. CPC C07D 221/08; A61K 31/473

(57) ABSTRACT

Described is a compound of Formula II1, a method of manufacturing thereof as well as uses thereof.

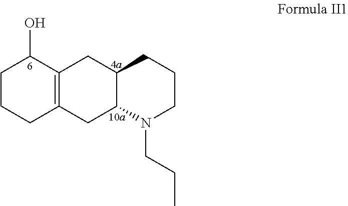 [SMILES: CCCN1CCC[C@@H]2CC3=C(CCCC3O)C[C@H]21]

### TECHNICAL FIELD

The present disclosure relates to novel 1,2,3,4,4a,5,6,7,8,9,10,10a-dodecahydrobenzo[g]quinolin-6-ol compounds, a method for preparing said compounds, a pharmaceutical composition comprising said compounds and to uses of said compounds.

### BACKGROUND

Neurodegenerative diseases and neurological disorders are becoming increasingly prevalent with the growing number of aging populations worldwide. One of the most common of these diseases and disorders is Parkinson's disease which is characterized by tremors, motor disturbances and coordination defects. Parkinson's disease is believed to be caused by deterioration of dopamine-producing neurons of the brain, in particular the substantia nigra neurons.

Currently there is no known cure for Parkinson's disease. Instead, the treatment of Parkinson's disease is focused on providing symptom relief.

The state of the art treatment of Parkinson's disease involves administering to the patient L-dopa or apomorphine. These compounds are known to exert their action by being agonists of the D1 and/or D2 dopamine receptors. In the case of L-dopa, it is its active metabolite dopamine that is the species that interacts at the D1 and/or D2 dopamine receptors. However, therapies involving L-dopa or apomorphine are associated with drawbacks. For instance, L-dopa has low and variable bioavailability which depends on protein intake. Further, use of L-dopa may result in long term complications such as dyskinesias. Apomorphine has a very short duration of action and a patient therefore has to take multiple injections per day. Apomorphine is also extensively metabolized and cannot be administered orally or intravenously. In fact, apomorphine only allows for subcutaneous administration such as via injection or infusion.

The low oral bioavailability of L-dopa and apomorphine is associated with the presence of a catechol moiety in these compounds. In order to reach the bloodstream and enable transport to the brain most of the pharmaceutical drug has to pass through the gastrointestinal tract and the liver, where most catecholamines are subjected to rapid biotransformation. By introducing protecting groups at the hydroxyl and/or the amino functions of the compound, the oral bioavailability can be increased by e.g. slowing down the transformation into the active metabolite and/or allowing the protected drug to function as a prodrug which may release the drug by removal of the protective group by cleavage. One such prodrug of dopamine is for instance docarpamine wherein the two hydroxyl groups of dopamine are protected as ethyl carbonate esters and its amino group is protected with an acetyl methionine moiety.

J. Med. Chem 2006, 49, 1494-1498, describes enone prodrugs of dopaminergic catecholamines in the research area of dopamine receptor agonists. It is disclosed that the (−)-enantiomer of the trans-isomer of the compound designated as 1-propyl-2,3,4,4a,5,7,8,9,10,10a-decahydro-1H-benzo[g]quinolin-6-one (also named Compound 4) acts as an enone prodrug of a dopamine receptor agonist. It is suggested that said enone compound is converted in vivo to the corresponding catechol compound designated as N-(n-propyl)-6,7-di-OH-benzo[g]quinoline (also named Compound 3).

Bioorganic & Medicinal Chemistry, 16 (2008), 3438-3444, discloses a synthesis and pharmacological evaluation of a compound titled as racemic trans-1-propyl-1,2,3,4,4a,5,10,10a-octahydrobenzo[g]quinoline-6,7-diol (also named Compound 4), which is believed to be the active form of its enone prodrug. It is stated that the catechol moiety of this compound is decisive for the low bioavailability observed. Further, it is stated that the high efficiency of the compound results in the possibility of administering low dose, which could make it a candidate for the treatment of Parkinson's disease.

WO 2010/097092 describes compounds for treating dyskinesia related disorders, such as Parkinson's disease. The compound (4aR,10aR)-1-n-propyl-1,2,3,4,4a,5,10,10a-octahydro-benzo[g]quinoline-6,7-diol (also named Compound 10) was found to be an active metabolite functioning as a potent agonist at both the D1 and D2 receptors in vitro and possessing a superior profile as a dopamine agonist in vivo. It is also described that the compound designated as (4aR,10aR)-1-n-propyl-2,3,4,4a,5,7,8,9,10,10a-decahydro-1H-benzo[g]quinolin-6-one (also named Compound 12) may be used for preparing the aforementioned metabolite as well as in the preparation of a medicament for treating Parkinson's disease while maintaining a low dyskinesia induction profile.

WO 2001/078713 discloses maleate salts of the two enantiomers of the compound designated as 1-propyl-trans-2,3,4,4a,5,7,8,9,10,10a-decahydrobenzo[g]quinoline-6-one.

WO 2019/101917 discloses catecholamine prodrugs for use in the treatment in Parkinson's disease. More specifically, it is stated that the invention relates to new prodrug derivatives of the compound (4aR,10aR)-1-n-Propyl-1,2,3,4,4a,5,10,10a-octahydro-benzo[g]quinoline-6,7-diol, and it is reported that glucuronide conjugates and sulfate conjugates of this compound are orally active prodrugs of this compound.

WO 2020/234270 and WO 2020/234271 both disclose processes for the manufacture of the catecholamine prodrug (2S,3S,4S,5R,6S)-3,4,5-trihydroxy-6-(((4aR,10aR)-7-hydroxy-1-propyl-1,2,3,4,4a,5,10,10a-octahydrobenzo[g]quinolin-6-yl)oxy)tetrahydro-2H-pyran-2-carboxylic acid. It is stated that said catecholamine prodrug is for use in the treatment of neurodegenerative diseases and disorders such as Parkinson's disease.

WO 2020/234272 discloses a new solid form of the catecholamine prodrug (2S,3S,4S,5R,6S)-3,4,5-trihydroxy-6-(((4aR,10aR)-7-hydroxy-1-propyl-1,2,3,4,4a,5,10,10a-octahydrobenzo[g]quinolin-6-yl)oxy)tetrahydro-2H-pyran-2-carboxylic acid. It is stated that said catecholamine prodrug is for use in the treatment of neurodegenerative diseases such as Parkinson's disease.

WO 2020/234273 discloses a process for the manufacture of the two compounds (6aR,10aR)-7-propyl-6,6a,7,8,9,10,10a, 11-octahydro-[1,3]dioxolo[4′.5′.5.6]benzo[1,2-g]quinoline and (4aR,10aR)-1-n-propyl-1,2,3,4,4a,5,10,10a-octahydro-benzo[g]quinoline-6,7-diol. It is stated that the compounds are for use in the treatment of neurodegenerative diseases such as Parkinson's disease.

WO 2020/234274, WO 2020/234275, WO 2020/234276, and WO 2020/234277 disclose different prodrugs of the catecholamine (4aR,10aR)-1-n-propyl-1,2,3,4,4a,5,10,10a-octahydro-benzo[g]quinoline-6,7-diol. The compounds are for use in the treatment of neurodegenerative or neuropsychiatric diseases such as Parkinson's disease.

The listing or discussion of an apparently prior-published document in this specification should not necessarily be taken as an acknowledgement that the document is part of the state of the art or is common general knowledge.

The prior art compound 1-propyl-trans-2,3,4,4a,5,7,8,9,10,10a-decahydro-1H-benzo[g]quinolin-6-one (hereinafter named as (4aR,10aR)-1-propyl-1,2,3,4,4a,5,8,9,10,10a-decahydrobenzo[g]quinolin-6 (7H)-one or (4aR,10aR)-1-propyl-1,2,3,4,4a,5,8,9,10,10a-decahydrobenzo[g]quinolin-6 (7H)-one), either as the racemate or as its (4aR,10aR)-enantiomer, is as mentioned above a prodrug of an extremely potent orally active dopamine receptor agonist. However, administration of these compounds is associated with a risk for quickly obtaining high peak plasma concentrations and/or emergent side effects such as nausea and vomiting.

Furthermore, in Parkinson's disease, degeneration of the nigro-striatal dopamine pathways is associated with the core motor symptoms. This deficit is addressed by available dopamine receptor agonists. However, there is also a degeneration of other dopaminergic pathways of the brain. In particular, degeneration of the mesolimbic dopamine pathways is associated with important non-motor symptoms such as depression and apathy in Parkinson's disease.

There is a need for novel therapeutic agents allowing for treatment of CNS diseases, disorders and/or conditions such as Parkinson's disease. In particular, there is a need for a therapeutic agent that is potent, has a long duration of action and/or has few side effects. Further, there is also a need for a therapeutic agent allowing for treating non-motor symptoms associated with Parkinson's disease.

### SUMMARY

It is an object of the present disclosure to provide novel therapeutically active compounds that at least partly overcome or mitigate some of the drawbacks of the aforementioned compounds. A further object is to provide novel therapeutically active compounds useful in the treatment of a CNS disease, disorder and/or condition such as Parkinson's disease. Still a further object of the present disclosure is to provide novel therapeutically active compounds that are potent, have a long duration of action and/or have few side effects, such as nausea and vomiting, when used in the treatment of a CNS disease, disorder and/or condition such as Parkinson's disease. It is also an object of the present disclosure to provide a novel therapeutic agent allowing for treating non-motor symptoms associated with Parkinson's disease. It is also an object of the present disclosure to provide aspects and/or advantages not provided by hitherto known techniques.

The present disclosure provides a compound of Formula II:

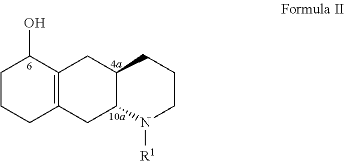

or a pharmaceutically acceptable salt thereof,
wherein
carbon 4a and carbon 10a both have R configuration, and
R¹ is methyl, ethyl or n-propyl.

The present disclosure also provides a pharmaceutical composition comprising a therapeutically acceptable amount of a compound of Formula II as described herein, or a pharmaceutically acceptable salt thereof, in admixture with at least one pharmaceutically acceptable carrier, excipient and/or diluent.

Further, there is also provided a compound of Formula II as described herein, or a pharmaceutically acceptable salt thereof, or a pharmaceutical composition as described herein for use as a medicament.

There is also provided a compound of Formula II as described herein, or a pharmaceutically acceptable salt thereof, or a pharmaceutical composition as described herein for use in the treatment of one or more of the following: Parkinson's disease, Huntington's disease, Restless leg syndrome, Alzheimer's disease, schizophrenia, attention deficit hyperactivity disorder, drug addiction.

The present disclosure also provides use of a compound of Formula II as described herein, or a pharmaceutically acceptable salt thereof, or
a pharmaceutical composition as described herein
for the manufacture of a medicament for use in the treatment of one or more of the following: Parkinson's disease, Huntington's disease, Restless leg syndrome, Alzheimer's disease, schizophrenia, attention deficit hyperactivity disorder, drug addiction.

The present disclosure also provides a method for treatment of one or more of the following:
Parkinson's disease, Huntington's disease, Restless leg syndrome, Alzheimer's disease, schizophrenia, attention deficit hyperactivity disorder, drug addiction,
said method comprising administering to a mammal, such as a human or an animal, in need thereof, an effective amount of (i) a compound of Formula II as described herein, or
a pharmaceutically acceptable salt thereof, or (ii) a pharmaceutical composition as described herein.

There is also provided a method for preparing a compound of Formula II as described herein, or a pharmaceutically acceptable salt thereof, said method comprising the steps of:
a) reducing a compound of Formula I

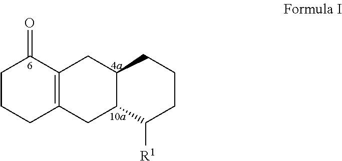

wherein R¹ is as described herein,
with a reducing agent in the presence of a Lewis acid and a solvent
whereby the carbonyl group is reduced into a hydroxyl group thereby providing the compound of Formula II, and
b) optionally separating the compound of Formula II into a compound of
Formula IIa and Formula IIb as described herein, and
c) optionally combining the compound of Formula II obtained in step a) or step b) with a pharmaceutically acceptable acid thereby providing a pharmaceutically acceptable salt of the compound of Formula II.

### BRIEF DESCRIPTION OF THE DRAWINGS

FIG. 1 shows a reaction scheme for the preparation of a compound of Formula II1.

FIG. 2 shows a chromatogram for the 6S and 6R epimers of (4aR,10aR)-1-propyl-1,2,3,4,4a,5,6,7,8,9,10,10a-dodecahydrobenzo[g]quinolin-6-ol.

FIG. 3 shows the contralateral turns as a function of time following intraperitoneal administration of apomorphine in a dose of 1 mg/kg.

FIG. 4 shows the contralateral turns as a function of time following intraperitoneal administration and peroral administration of the 6S and 6R epimers of (4aR,10aR)-1-propyl-1,2,3,4,4a,5,6,7,8,9,10,10a-dodecahydrobenzo[g]quinolin-6-ol.

FIG. 5 shows effects on gene expression (mRNA) of Arc in different brain regions by the prior art compound according to Preparation 4A and by the compound according to Example 4B.

FIG. 6 shows effects on locomotor activity for the prior art compound according to Preparation 4A when said compound was administered subcutaneously.

FIG. 7 shows effects on locomotor activity for the compound of the present disclosure according to Example 4B when said compound was administered subcutaneously.

### DESCRIPTION

The present disclosure provides a compound of Formula II:

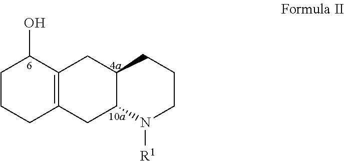

or a pharmaceutically acceptable salt thereof,
wherein
carbon 4a and carbon 10a both have R configuration, and
R¹ is methyl, ethyl, or n-propyl.

R¹ of the compounds described herein may be methyl, ethyl or n-propyl. For instance, R¹ may be ethyl or n-propyl. In an example, R¹ is ethyl. In a further example, R¹ is n-propyl.

The compound of Formula II may exist as a compound of Formula IIa and/or a compound of Formula IIb:

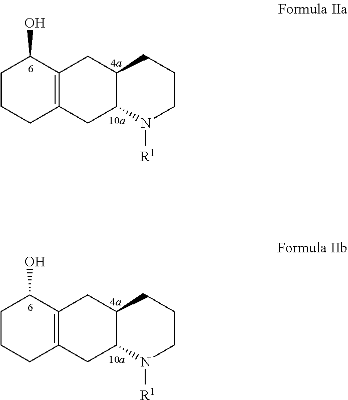

It will be appreciated that R¹ for the compounds of Formula IIa and Formula IIb, respectively, may be as described herein. Further, it is understood that the compounds of Formula IIa and Formula IIb are epimers that differ in configuration on carbon 6 (i.e. the carbon linked to the OH group). The compound of Formula IIa is the 6R epimer while the compound of Formula Ib is the 6S epimer. For the compounds of Formula IIa and Formula IIb the stereochemistry of the tricyclic system is as depicted herein, i.e. the ring containing the nitrogen exhibits trans configuration wherein the carbon 4a has R configuration and the carbon 10a has R configuration. The numbering of the carbon atoms for the compound of Formula II is shown below.

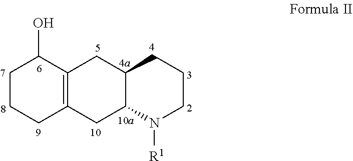

In an example, the compound of the present disclosure is a compound of Formula IIa. In a further example, the compound of the present disclosure is a compound of Formula IIb. In still a further example, the compound of Formula II is a mixture of the compound of Formula IIa and the compound of Formula IIb, such as a 1:1 mixture of the compound of Formula IIa and the compound of Formula IIb.

It will be appreciated that the nitrogen atom of the compounds disclosed herein may be provided in oxidized form such as a compound of Formula II2. Persons skilled in the art will understand that such compounds may be administered to a patient or formed in vivo after administration to a patient.

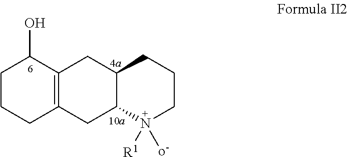

The R¹ group of the compound of Formula II may be n-propyl thereby providing a compound of Formula II1, Formula IIa1 and/or Formula IIb1.

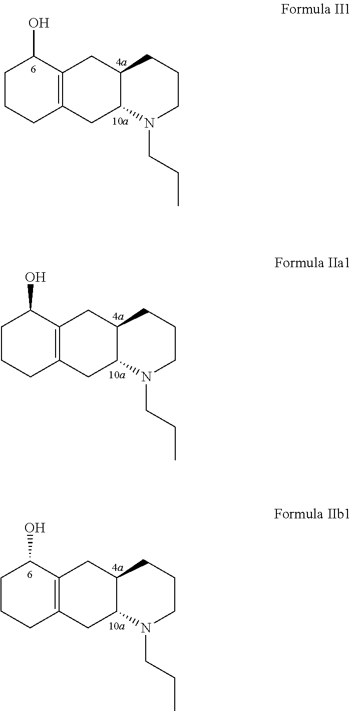 [SMILES: CCCN1CCC[C@@H]2CC3=C(CCCC3O)C[C@H]21 ; CCCN1CCC[C@@H]2CC3=C(CCC[C@H]3O)C[C@H]21 ; CCCN1CCC[C@@H]2CC3=C(CCC[C@@H]3O)C[C@H]21]

The chemical name of the compound of Formula II1 may be (4aR,10aR)-1-propyl-1,2,3,4,4a,5,6,7,8,9,10,10a-dodecahydrobenzo[g]quinolin-6-ol.

The chemical name of the compound of Formula IIa1 may be: (4aR,6R,10aR)-1-propyl-1,2,3,4,4a,5,6,7,8,9,10,10a-dodecahydrobenzo[g]quinolin-6-ol.

The chemical name of the compound of Formula IIb1 may be: (4aR,6S,10aR)-1-propyl-1,2,3,4,4a,5,6,7,8,9,10,10a-dodecahydrobenzo[g]quinolin-6-ol.

The compounds described herein may be provided as a single diastereomer such as a diastereomer essentially free of any other diastereomer. The single diastereomer may have R configuration at carbon 6, and have R configuration both at carbon 4a and at carbon 10a. Alternatively, the single diastereomer may have S configuration at carbon 6, and have R configuration both at carbon 4a and at carbon 10a. Further, the single diastereomer may be provided in a diastereomeric excess equal to or above 90%, such as equal to or above 95%, or such as equal to or above 99%. As used herein, the diastereomeric excess equals % (i.e. percentage) of the major diastereomer minus % of the minor diastereomer. For instance, a mixture composed of 95% of the major diastereomer and 5% of the minor diastereomer has a diastereomeric excess of 90%.

The present disclosure also provides a pharmaceutically acceptable salt of the compound(s) described herein, such as the compounds of Formula II, Formula IIa, Formula IIb, Formula II2, Formula II1, Formula IIa1 and Formula IIb1.

The pharmaceutically acceptable salt of the compound(s) described herein may be provided as a combination of a compound of Formula II as described herein and an organic acid. Further, the pharmaceutically acceptable salt of the compound(s) described herein may be provided as a combination of a compound of Formula II as described herein and an organic acid in a ratio of 1:1, 2:1 or 1:2. In an example, the ratio is 1:1. In a further example, the ratio is 2:1 In still a further example, the ratio is 1:2. The organic acid may be D-tartaric acid.

For instance, there is provided a salt of Formula II11

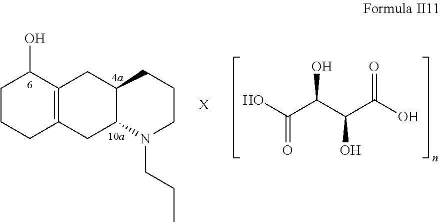

said salt being a combination of the compound of Formula II1 and D-tartaric acid:

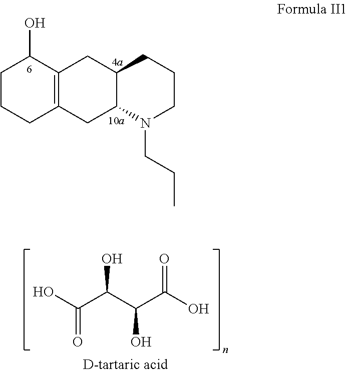

in a ratio of 1: n,
wherein
n is 1, 0.5 or 2. In particular, n may be 2.

Further, there is provided a salt of Formula IIa11

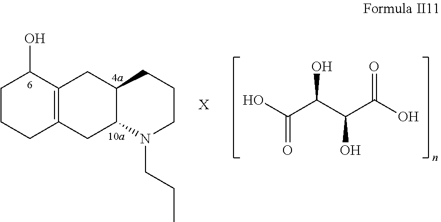

said salt being a combination of the compound of Formula IIa1 and D-tartaric acid:

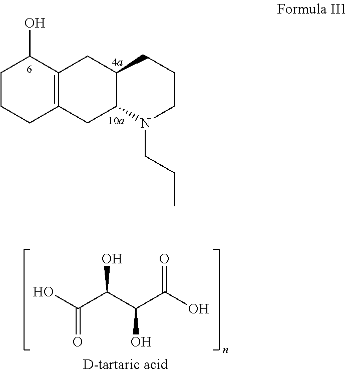

in a ratio of 1: n,
wherein
n is 1, 0.5 or 2. In particular, n may be 2.

There is also provided a salt of Formula IIb11

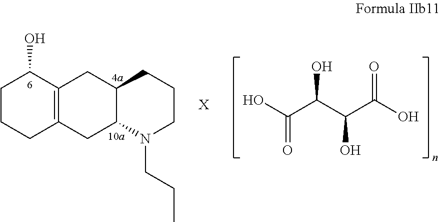

said salt being a combination of the compound of Formula IIb1 and D-tartaric acid:

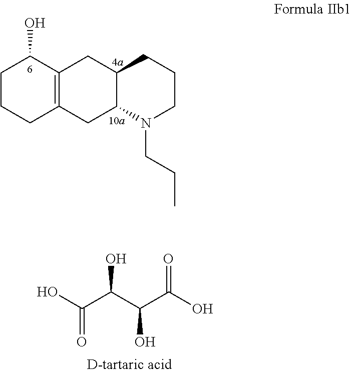 [SMILES: CCCN1CCC[C@@H]2CC3=C(CCC[C@@H]3O)C[C@H]21 ; O=C(O)[C@@H](O)[C@H](O)C(=O)O]

in a ratio of 1: n,
wherein
n is 1, 0.5 or 2. In particular, n may be 2.

It will be appreciated that the salt(s) described herein may be provided as a co-crystal. Thus, there is provided a co-crystal of a salt as described herein. For instance, there is provided a co-crystal of D-tartaric acid and a salt as described herein, such as a salt of Formula II11, Formula IIa11, Formula IIb11. In a further example, there is provided a co-crystal of (i) D-tartaric acid and (ii) a salt of Formula IIa11 wherein n is 1 resulting in a ratio of D-tartaric acid and a compound of Formula IIa1 of 2:1 in said co-crystal.

The compounds described herein, or a pharmaceutically acceptable salt thereof, may exist in solid form, i.e. they may be provided as a solid. For example, the compounds described herein, or a pharmaceutically acceptable salt thereof, may be amorphous, crystalline or a mixture thereof. Further, the compounds described herein, or a pharmaceutically acceptable salt thereof, may exist in crystalline form, i.e. they may be provided as crystal(s). The degree of crystallinity may be equal to or above 80%, 85%, 90%, 95% or 99%.

The compound of Formula II described herein, or a pharmaceutically acceptable salt thereof, may be included in a pharmaceutical composition. Thus, there is provided a pharmaceutical composition comprising a therapeutically acceptable amount of a compound of Formula II as described herein, or a pharmaceutically acceptable salt thereof, in admixture with at least one pharmaceutically acceptable carrier, excipient and/or diluent. As used herein, the expression “therapeutically effective amount” means an amount of a compound as disclosed herein that is sufficient to induce the desired therapeutic effect in a patient to which the compound is administered. The pharmaceutical composition may be for oral administration. Additionally or alternatively, the pharmaceutical composition may be for rectal, intracisternal, intravaginal, intraperitoneal and/or parenteral administration. In an example, the parenteral administration may be intravenous, intramuscular or subcutaneous administration. Further, the pharmaceutical composition may be provided in solid form such as in the form of one or more capsules, tablets, pills, powders and/or granules. Alternatively, the pharmaceutical composition may be provided in liquid form such as in the form of one or more emulsion(s), solution(s), suspension(s) and/or syrup(s).

There is also provided a compound of Formula II as described herein, or a
pharmaceutically acceptable salt thereof, or
a pharmaceutical composition as described herein
for use as a medicament.

There is also provided a compound of Formula II as described herein, or a pharmaceutically acceptable salt thereof,
or a pharmaceutical composition as described herein
for use in the treatment of one or more of the following:
Parkinson's disease, Huntington's disease, Restless leg syndrome, Alzheimer's disease, schizophrenia, attention deficit hyperactivity disorder (ADHD), drug addiction. For instance, the treatment may comprise or consist of treatment of Parkinson's disease. In a further example, the treatment may comprise or consist of treatment of Huntington's disease or Restless leg syndrome. In still a further example, the treatment may comprise or consist of treatment of Alzheimer's disease or schizophrenia. In yet an example, the treatment may comprise or consist of treatment of attention deficit hyperactivity disorder (ADHD) or drug addiction.

As used herein, the term treatment may involve one or more of the following: therapeutic treatment, palliative treatment, treatment reducing worsening or the development of a disorder or disease as described herein. For instance, the treatment may be therapeutic treatment and/or palliative treatment. Additionally or alternatively, the treatment may be a treatment reducing worsening or the development of a disorder or disease as described herein.

The present disclosure also provides use of a compound of Formula II as described herein, or a pharmaceutically acceptable salt thereof,
or a pharmaceutical composition as described herein
for the manufacture of a medicament for use in the treatment of one or more of the following: Parkinson's disease, Huntington's disease, Restless leg syndrome, Alzheimer's disease, schizophrenia, attention deficit hyperactivity disorder, drug addiction. For instance, the treatment may comprise or consist of treatment of Parkinson's disease. In a further example, the treatment may comprise or consist of treatment of Huntington's disease or Restless leg syndrome. In still a further example, the treatment may comprise or consist of treatment of Alzheimer's disease or schizophrenia. In yet an example, the treatment may comprise or consist of treatment of attention deficit hyperactivity disorder (ADHD) or drug addiction.

The present disclosure also provides a method for treatment of one or more of the following:
Parkinson's disease, Huntington's disease, Restless leg syndrome, Alzheimer's disease, schizophrenia, attention deficit hyperactivity disorder, drug addiction,
said method comprising administering to a mammal, such as a human or an animal, in need thereof, an effective amount, i.e. a therapeutically effective amount, of (i) a compound of Formula II as described herein, or a pharmaceutically acceptable salt thereof, or (ii) a pharmaceutical composition described herein. For instance, the disease, condition and/or disorder may involve Parkinson's disease. Thus, there is provided a method for treatment of Parkinson's disease comprising administering to a mammal, such as a human or an animal, in need thereof, an effective amount of (i) a compound of Formula II as described herein, or a pharmaceutically acceptable salt thereof, or (ii) a pharmaceutical composition as described herein. For instance, the treatment may comprise or consist of treatment of Parkinson's disease. In a further example, the treatment may comprise or consist of treatment of Huntington's disease or Restless leg syndrome. In still a further example, the treatment may comprise or consist of treatment of Alzheimer's disease or schizophrenia. In yet an example, the treatment may comprise or consist of treatment of attention deficit hyperactivity disorder (ADHD) or drug addiction.

As used herein, Parkinson's disease includes motor symptoms with or without non-motor symptoms. The main motor symptoms include tremor, rigidity, slowness of movement and difficulty in walking. Collectively, these main motor symptoms are known as “parkinsonism” or “parkinsonian syndrome”. Non-motor symptoms include cognitive functional decline, depression, anxiety, apathy and/or dementia such as Parkinson's disease dementia. Examples of cognitive functional decline include problems with memory, language, thinking, learning and/or judgment.

The treatment of Parkinson's disease described herein may take place without or substantially without inducing side effects such as nausea or vomiting. Thus, the treatment may be associated with no or mild side effects such as nausea and/or vomiting.

The present disclosure also provides a method for preparing a compound of Formula II as described herein, or a pharmaceutically acceptable salt thereof, said method comprising the steps of:
a) reducing a compound of Formula I

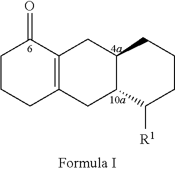

wherein R¹ is as described herein,
with a reducing agent in the presence of a Lewis acid and a solvent
whereby the carbonyl group is reduced into a hydroxyl group thereby providing the compound of Formula II, and
b) optionally separating the compound of Formula II into a compound of
Formula IIa and Formula IIb as described herein, and
c) optionally combining the compound of Formula II obtained in step a) or step b) with a pharmaceutically acceptable acid thereby providing a pharmaceutically acceptable salt of the compound of Formula II.

The pharmaceutically acceptable acid in step c) may be a pharmaceutically acceptable acid as described herein. In particular, the pharmaceutically acceptable acid may be D-tartaric acid.

For instance, R¹ may be n-propyl in the compound of Formula I thereby providing a compound of Formula I1.

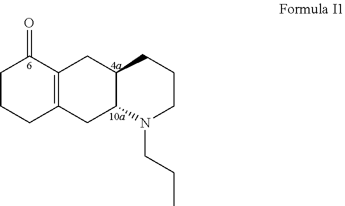 [SMILES: CCCN1CCC[C@@H]2CC3=C(CCCC3=O)C[C@H]21]

In the method for preparing the compound of Formula II the reducing agent, the Lewis acid and the solvent may be selected so as to reduce the carbonyl selectively into a hydroxyl group, i.e. the carbonyl group is reduced into a hydroxyl group without or substantially without reducing the double bond into a single bond. The reducing agent may be a hydride such as sodium borohydride, and/or the Lewis acid may comprise cerium (III) chloride such as cerium (III) chloride heptahydrate, and/or the solvent may comprise a protic solvent such as an alcohol such as methanol. This will generally provide a 1:1 mixture of compounds of Formula IIa and Formula IIb. A chiral reducing agent may be used if it is desired to produce an excess of the compound of Formula IIa or the compound of Formula IIb. For example, a chiral alkylborohydride such as diisopinocampheylborane may be used. Methods commonly known in the art may be used in order to obtain the compound of Formula IIa or the compound of Formula IIb in chemically and/or stereochemically pure form.

### Pharmaceutically Acceptable Salts

Compounds of the present disclosure may be provided in the form of a pharmaceutically acceptable salt. As used herein “pharmaceutically acceptable salt”, where such salt is possible, includes salt prepared from a pharmaceutically acceptable non-toxic acid, i.e.

pharmaceutically acceptable acid addition salts. The pharmaceutically acceptable salt may be formed by combining a compound as described herein with an organic acid or inorganic acid in a desired ratio using e.g. methods known in the art. Thus, the pharmaceutically acceptable salt may be a combination of the compound of Formula I and an acid such as a combination of the compound of Formula II and an acid in a ratio of 1:1, 1:2 or 2:1.

Examples of pharmaceutically acceptable salts include, without limitation, non-toxic inorganic and organic acid addition salts such as hydrochloride, hydrobromide, borate, nitrate, perchlorate, phosphate, sulphate, formate, acetate, ascorbate, benzenesulphonate, benzoate, cinnamate, citrate, embonate, enantate, fumarate, glutamate, glycolate, lactate, maleate, malonate, mandelate, methanesulphonate, naphthalene-2-sulphonate, phthalate, propionate, salicylate, sorbate, stearate, succinate, tartrate, toluene-p-sulphonate, and the like.

Other acids such as oxalic acid, may be useful in the preparation of salts useful as intermediates in obtaining a compound of the present disclosure and its pharmaceutically acceptable acid addition salt.

### Solvates

It is to be understood that compounds of the present disclosure, or a pharmaceutically acceptable salt thereof, may exist in solvated form. Alternatively, the compounds of the present disclosure, or a pharmaceutically acceptable salt thereof, may exist in non-solvated forms. The term “solvate” is used herein to describe a molecular complex comprising a compound of the present disclosure and one or more pharmaceutically acceptable solvent molecule(s). The term “hydrate” is employed when the solvent is water. Thus, solvated forms may include hydrated forms such as monohydrate, dihydrate, hemihydrate, trihydrate, tetrahydrate, and the like.

### Polymorphs

The compounds of the present disclosure, or a pharmaceutically acceptable salt thereof, may exist in a continuum of solid states ranging from fully amorphous to fully crystalline. Thus, it is to be understood that the compounds of the present disclosure may be in the form of a polymorph.

### Labelled Compounds

The compounds of the present disclosure, or a pharmaceutically acceptable salt thereof, may be used in their labelled or unlabelled form. In the context of this present disclosure the labelled compound has one or more atoms replaced by an atom having an atomic mass or mass number different from the atomic mass or mass number usually found in nature. The labelling may allow easy quantitative detection of said compound.

For example, there is provided a compound as described herein which is labelled with one or more isotopes, such as for example tritium (³H), deuterium (²H) or carbon-14 (¹⁴C). In an example, the compound is labelled with one or more deuterium atoms. All isotopic variations of the compounds of the present disclosure, whether radioactive or not, are intended to be encompassed within the scope of the present disclosure.

Thus, the present disclosure provides a compound as described herein which is labelled with one or more isotopes such as deuterium. The compounds labelled with an isotope as described herein may be combined with an acid as described herein thereby providing a salt such as a pharmaceutically acceptable salt as described herein.

### Dosage

It will be appreciated that the compounds described herein may be administered in a therapeutically acceptable amount. For example, the dose may be from about 0.0001 mg/kg bodyweight to about 5 mg/kg bodyweight, such as from 0.001 mg/kg bodyweight to about 1 mg/kg bodyweight. The exact dosages will depend upon the frequency and mode of administration, the sex, the age, the weight, and the general condition of the subject to be treated, the nature and the severity of the condition to be treated, any concomitant diseases to be treated, the desired effect of the treatment and/or other factors known to those skilled in the art.

### Methods of Preparation

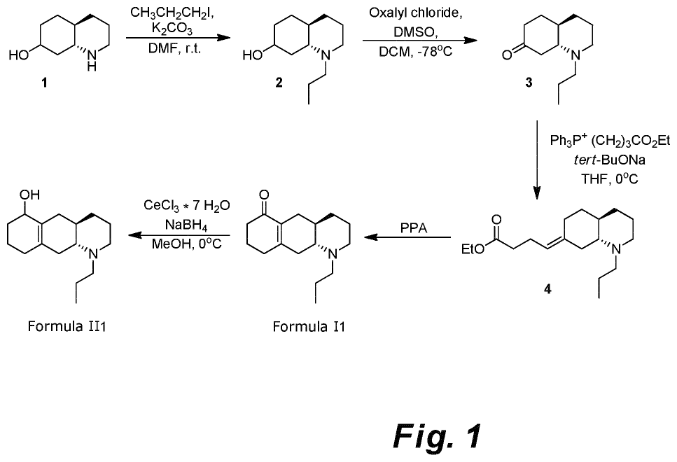

Compounds of the present disclosure may be prepared as described herein. For instance, the compounds of the present disclosure may be prepared as shown in FIG. 1.

The end products of the reactions described herein may be isolated by conventional techniques, e.g. by extraction, crystallization, distillation, chromatography, etc. The compounds of the present disclosure may be prepared in chemically pure form, i.e. they are substantially free from reactants, solvents, impurities etc. Further, the compounds of the present disclosure may be prepared in substantially stereochemically pure form. For instance, the compound of Formula II may contain the compound of Formula IIa and the compound of Formula IIb in a ratio equal to or above 95:5, 96:4, 97:3, 98:2 or 99:1. In a further example, the compound of Formula II may contain the compound of Formula IIb and the compound of Formula IIa in a ratio equal to or above 95:5, 96:4, 97:3, 98:2 or 99:1.

Persons skilled in the art will appreciate that, in order to obtain compounds of the present disclosure the individual process steps mentioned hereinbefore may be performed in a different order, and/or the individual reactions may be performed at different stage in the overall route (i.e. chemical transformations may be performed upon different intermediates to those associated hereinbefore with a particular reaction).

The disclosure is illustrated in the following non-limitative Examples.

### EXAMPLES

The naming of the compounds (preferred IUPAC name) as disclosed herein was made using ChemDraw Ultra, version 12.0.2. 1076. In this document, if the chemical name and the chemical structure are inconsistent the chemical structure should be considered to be the correct structure.

### General Methods

Chemicals were mainly purchased from Sigma-Aldrich. Preparative HPLC was performed on a Gilson system equipped with a UV detector. For flash chromatography, Biotage Isolera Vers 1.2 with Sfar Silica HC D columns were used. Analytical HPLC/LCMS was performed using an Agilent 1100 series Liquid Chromatography/Mass Selective Detector (MSD) (Single Quadropole) equipped with an electrospray interface and a UV diode array detector. Analyses were performed by using either an ACE 3 C8 (3.0×50 mm) column with a 10-97% gradient of acetonitrile in 0.1% aqueous TFA over 3 min and a flow of 1 mL/min, or an Xbridge C18 (3.0×50 mm) column with a 10-97% gradient of acetonitrile in 10 mM ammonium bicarbonate over 3 min and a flow of 1 mL/min and UV detection. Alternatively, a 10-100% gradient of acetonitrile in 0.03% acetic acid was used as eluent.

Low resolution mass spectra were recorded on a HP 5970A instrument operating at an ionization potential of 70 eV. The mass detector was interfaced with a HP5700 gas chromatograph equipped with a HP-5 MS UI GC column (15 m, 0.25 mm, 0.25 μm) with a helium gas flow of 40 cm/s.

Optical measurements were performed at 25° C. using a sodium lamp (A=589 nM). 1H-NMR spectra were recorded on a Varian 400 MHz instrument at 25° C. or on a Bruker 700 or 800 MHz instrument when specified. 13C NMR spectrum was obtained using a Bruker instrument operating at 200 MHz.

Melting points were determined by a Buchi B-545 apparatus and are uncorrected.

### Abbreviations

AP anterior posterior
aq. Aqueous
ARC activity-regulated cytoskeleton-related protein
Ctrl Control
d doublet
dd doublet of doublets
DCM dichloromethane
DEA diethylamine
DMF dimethylformamide
DMSO dimethsulphoxide
egr-1 early growth response protein 1
EtOAc ethyl acetate
EtOH ethanol
g gram(s)
mg milligram(s)
kg kilogram(s)
i.p. intraperitoneal
IP intraperitoneal
IUPAC International Union of Pure and Applied Chemistry
HPLC High Performance Liquid Chromatography
HPRT Hypoxanthine-guanine phosphoribosyltransferase
M molar, i.e. mole(s)/liter
MeOH methanol
millimolar, i.e. millimole(s)/liter mM
ML medial lateral
MS(ESI+) Mass Spectroscopy Electrospray Ionization
min. minute(s)
mg milligram(s)
mL millilitre(s)
ml millilitre(s)
mol mole
mmol (e) millimole
MS Mass Spectrometry
NMR Nuclear Magnetic Resonance
Npas4 Neuronal PAS Domain Protein 4
OHDA hydroxydopamine
PCR Polymerase Chain Reaction
PPA polyphosphoric acid
PO perorally
RT Reverse transcriptase
r.t. room temperature, i.e. from about 20° C. to about 25° C. such as about 22° C.
SEM standard error of the mean
SC subcutaneously
td triplet of doublets
THF Tetrahydrofurane
UV ultraviolet
V ventral
Å Angstrom

### Example 1

### Synthesis of (4aR,10aR)-1-propyl-1,2,3,4,4a,5,8,9,10,10a-decahydrobenzo[g]quinolin-6 (7H)-one (synthetic intermediate as well as prior art compound; compound of Formula II)

A) trans-1-propyldecahydroquinolin-7-ol (compound 2 in the synthesis of FIG. 1).

To a stirred solution of trans-decahydroquinolin-7-ol (1.32 mL, 12.9 mmol) in DMF (20 mL) was added 1-iodopropane (2.3 g, 13.5 mmol). After 10 min, potassium carbonate (8.9 g, 64 mmol) was added. The reaction mixture was stirred overnight and was then diluted with 80 mL of water and extracted with 3×30 mL of DCM. The organic layers were combined, washed with brine (4×40 mL) and concentrated to give trans-1-propyldecahydroquinolin-7-ol as a yellow oil (2.5 g, 12.7 mmol, 98% yield).

B) trans-1-propyloctahydroquinolin-7 (1H)-one (compound 3 in the synthesis of FIG. 1).

To a stirred solution of oxalyl chloride (2.07 ml, 24 mmol) in DCM (40 mL) at −78° C. was added DMSO (3.5 mL, 49 mmol) dropwise and the reaction mixture was stirred for 15 min at this temperature. Then, a solution of trans-1-propyldecahydroquinolin-7-ol (2.5 g, 12.7 mmol) in 10 mL of DCM was added at −78° C. and the reaction mixture was stirred for 1 h. Triethylamine (14 mL, 101 mmol) was added and the reaction mixture was slowly warmed to room temperature and stirred for 2 h. After this, the reaction mixture was washed with saturated NaHCO₃, water and brine. The organic phase was dried over Na₂SO₄ and concentrated to give trans-1-propyloctahydroquinolin-7 (1H)-one as a yellow oil (1.63 g, 8.3 mmol, 66% yield).

C) (E)-ethyl 4-(trans-1-propyloctahydroquinolin-7 (1H)-ylidene) butanoate (compound 4 in the synthesis of FIG. 1).

To a stirred suspension of (4-ethoxy-4-oxobutyl)triphenylphosphonium bromide (9.93 g, 22 mmol) in THF (50 mL) at 0° C. was added sodium tert-butoxide (2M in THF, 12.5 mL, mmol) and the reaction mixture was stirred for 30 min. Then, trans-1-propyloctahydroquinolin-7 (1H)-one (2.12 g, 10.9 mmol) in THF (10 mL) was added dropwise at 0° C. during 3 min and the reaction mixture was stirred over two days (until starting material disappeared). The reaction mixture was diluted with cold water (50 mL) with ice bath cooling and extracted with hexane (4×50 mL). The solvent was removed in vacuo, and the residue was triturated with 50 mL of hexane whereby a precipitate of Ph₃PO was formed. The precipitate was removed by filtration and the solution was concentrated to give 3.18 g of crude product (purity approximately 50%). The material was used in the next step without additional purification.

D) (4aR,10aR)-1-propyl-1,2,3,4,4a,5,8,9,10,10a-decahydrobenzo[g]quinolin-6 (7H)-one, starting material for the synthesis of the compound of Formula II1, which may also be prepared as in Reference 1.

A solution of (E)-ethyl 4-(trans-1-propyloctahydroquinolin-7 (1H)-ylidene) butanoate (3.18 g, 5.42 mmol) in DCM (3 mL) was added dropwise to PPA (30 g) at 100° C. during 5 min (temperature should not be higher than 110° C.) and followed by stirring for 2 h under heating. Then, ice and 50 ml of water were added and the mixture was extracted with DCM to remove impurities. The aqueous phase was basified with NH₃ (28% aq.) and extracted with DCM to give a crude mixture of the two isomers. This was purified by column chromatography (silica, gradient of 1 to 9% MeOH in DCM, with the MeOH containing 1% NH₃ to give the title compound (860 mg, 30% yield over 2 steps). MS (ESI+) m/z 248 [M+H]+. This mixture of enantiomers was separated using a preparative HPLC system equipped with a semipreparative chiral column (CHIRALPAK ID, 5 μm, 10×250 mm) and a mobile phase of heptane/isopropanol/diethylamine 80/20/0.1 with a flow rate of 4.6 mL/min.

### Peak 1:

(4aR,10aR)-1-propyl-1,2,3,4,4a,5,8,9,10,10a-decahydrobenzo[g]quinolin-6 (7H)-one (360 mg, 1.45 mmol). Optical measurement: [α]_{D}²⁵=−234° (c=0.035, methanol).

### Peak 2:

(4aS,10aS)-1-propyl-1,2,3,4,4a,5,8,9,10,10a-decahydrobenzo[g]quinolin-6 (7H)-one (400 mg, 1.62 mmol). Optical measurement: [α]_{D}²⁵=+228° (c=0.032, methanol).

### Example 2

### Synthesis of (4aR,10aR)-1-propyl-1,2,3,4,4a,5,6,7,8,9,10,10a-dodecahydrobenzo[g]quinolin-6-ol (Mixture of 6R and 6S epimers, i.e. compound of Formula II1 in the synthesis of FIG. 1)

Cerium (III) chloride heptahydrate (547 mg, 1.47 mmol) was added to a stirred and cooled (0° C.) solution of (4aR,10aR)-1-propyl-1,2,3,4,4a,5,8,9,10,10a-decahydrobenzo[g]quinolin-6 (7H)-one (300 mg, 1.21 mmol) in MeOH (8 mL) and stirred 15 min. Sodium borohydride (138 mg, 3.6 mmol) was added in 3 portions over 15 min, and after 1 h the reaction was completed. Water (10 mL) was added to the resulting white slurry and stirred 20 min. The clear aqueous solution was extracted with EtOAc (4×30 mL) and concentrated to give 190 mg of product as a mixture of diastereomers.

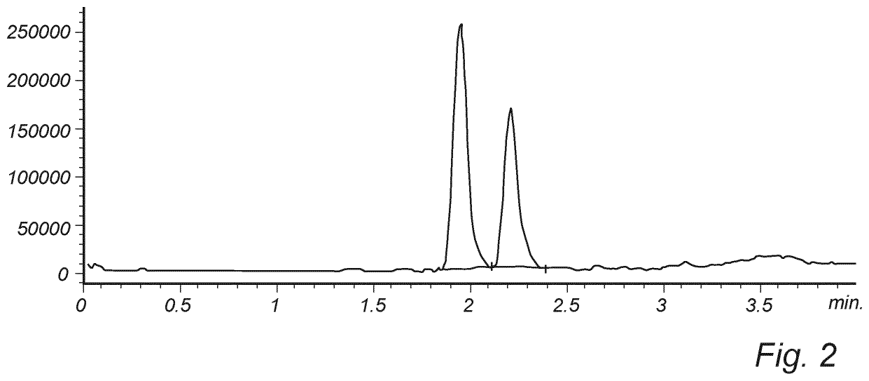

The two diastereomers (epimer 1 and epimer 2) were first separated in analytical scale, which is shown in the chromatogram of FIG. 2 where epimer 1 has the retention time 1.95 min and epimer 2 has 2.21 min under the following conditions: (Xbridge C18, 50×3.0, 3.5 u, 10 to 97% acetonitrile in 10 mM NH₄HCO₃ (pH 10) over 3 min, 1 ml/min.).

The mixture of diastereomers were also, in another experiment, separated using preparative HPLC (XBridge™ Prep C18 5 p.m OBD™ 19×50 mm column, 15 to 40% acetonitrile in 50 mM aqueous NH₄HCO₃ over 6 min). The two epimers were eluted in the same order as in the analytical experiment. One of these is the 6S epimer and the other is the 6R epimer, but at first it was not clear which epimer that elutes first. An X ray analysis then showed that the epimer that elutes first has 6R configuration and the epimer that elutes last has 6S configuration. The yield of Epimer 1 was 110 mg, 36.4% yield, and the yield of epimer 2 was 35 mg, 11.6% yield.

Final evaporation and drying gave two products as pale yellow oils which solidified overtime. The isomers were numbered by the order of elution from the column.

### Example 3

### NMR Spectra of the Compounds of Example 2.

### Mixture of Epimer 1 and Epimer 2:

¹H NMR (400 MHZ, CDCl₃) δ 4.01 (br s, 0.7H), 3.85 (br s, 0.3H), 2.94 (br d, J=11.2 Hz, 1H), 2.56-2.72 (m, 1H), 2.08-2.42 (multiplets), 0.93-1.09 (m, 1H), 0.85 (t, J=7.4 Hz, 3H). The total yield was 48%.

Epimer 1 (6R epimer):

¹H NMR (400 MHZ, CDCl₃): δ 6 4.02 (br s, 1H), 2.95 (br d, J=11.4 Hz, 1H), 2.59-2.70 (m, 1H), 2.30-2.41 (m, 1H), 2.12-2.29 (m, 2H), 1.28-2.09 (multiplets), 0.96-1.08 (m, 1H), 0.86 (t, J=7.3 Hz, 3H). MS(ESI+) m/z 250 [M+H]+.

Epimer 2:1 (6S epimer):

¹H NMR (400 MHZ, CDCl₃) δ 3.86 (br s, 1H), 2.95 (br d, J=10.2 Hz, 1H), 2.60-2.72 (m, 1H), 2.30-2.43 (m, 2H), 2.12-2.27 (m, 2H), 1.39-2.04 (multiplets), 0.93-1.08 (m, 1H), 0.86 (t, 1=7.4 Hz, 3H). MS(ESI+) m/z 250 [M+H)+.

### Example 4A

### Preparation 4A (4aR,10aR)-1-propyl-1,2,3,4,4a,5,8,9,10,10a-decahydrobenzo[g]quinolin-6 (7H)-one (Synthetic Intermediate as Well as Prior Art Compound, Compound of Formula II)

(E)-ethyl 4-(trans-1-propyloctahydroquinolin-7 (1H)-ylidene) butanoate (4.0 g, 13.7 mmol), which had been obtained in a similar fashion as in Example 1C, was mixed with Eaton's reagent (phosphorus pentoxide, 7.7 wt. % in methanesulfonic acid, 21.1 g) and the mixture was heated at 80° C. for 3 h. The reaction mixture was carefully added dropwise to an ice-cooled aqueous solution of NaHCO₃ (10%). After extracting three times with DCM, the organic solution was dried over Na₂SO₄, filtered and evaporated. The residue was purified by silica-gel chromatography using isooctane/EtOAc/MeOH (gradient, 0-100% EtOAc and then 0-100% MeOH) as eluent. There was obtained 1.4 g of the racemic intermediate and the two enantiomers were thereafter separated on a chiral column (Chiralpak IG, 10 mm×250 mm) using heptane, EtOH and DEA (90:10:0.1) as eluent. Approximately 25 mg of the racemate was each time loaded on the column and the isomer that eluted last from the column was collected. After pooling the desired fractions, the product was again purified by silica-gel chromatography using isooctane/EtOAc/MeOH as eluent (gradient, 0-100% EtOAc and then 0-100% MeOH). There was obtained 0.35 g of (4aR,10aR)-1-propyl-1,2,3,4,4a,5,8,9,10,10a-decahydrobenzo[g]quinolin-6 (7H)-one in its non-salt form as an oil. [α]²⁰_{D}=−215.6 (c, 0.010 g/mL, MeOH). ¹H NMR (800 MHZ, CDCl₃): δ 0.88 (t, 3H), 1.04 (m, 1H), 1.38 (m, 1H), 1.49 (m, 2H), 1.62 (m, 1H), 1.68 (m, 2H), 1.84 (m, 1H), 1.98 (m, 3H), 2.16 (m, 2H), 2.26 (m, 2H), 2.35 (m, 2H), 2.42 (m, 1H), 2.52 (m, 2H), 2.67 (m, 1H), 2.97 (m, 1H). The HCl-salt of (4aR,10aR)-1-propyl-1,2,3,4,4a,5,8,9,10,10a-decahydrobenzo[g]quinolin-6 (7H)-one was prepared by mixing 518 mg (2.1 mmol) of its non-salt form (synthesized in a similar fashion as above) with HCl in ethanol (1.25 M, 4 mL) and then concentrating the formed solution on a rotavapor. The residue was co-evaporated with ethanol and then crystallized from ethanol/diethyl ether. There was obtained 355 mg (60%) of the title compound as a white powder. Melting point: 220.7° C. [α]²⁰_{D}=−199,6 (c, 0.010 g/mL, MeOH). ¹H NMR (800 MHZ, methanol-d₄): δ 1.07 (t, 3H), 1.40 (m, 1H), 1.77 (m, 1H), 1.86 (m, 3H), 1.9-2.1 (m, 4H), 2.4-2.5 (m, 4H), 2.56 (m, 1H), 2.68 (m, 1H), 2.92 (m, 1H), 3.10 (m, 2H), 3.23 (m, 1H), 3.32 (m, 1H), 3.62 (m, 1H).

### Example 4B

### Epimer 1 of (4aR,10aR)-1-propyl-1,2,3,4,4a,5,6,7,8,9,10,10a-dodecahydrobenzo[g]quinolin-6-ol

(4aR,10aR)-1-propyl-1,2,3,4,4a,5,8,9,10,10a-decahydrobenzo[g]quinolin-6 (7H)-one in its non-salt form from Preparation 4A (0.33 g, 1.33 mmol) was dissolved in MeOH (8 mL) and cerium (III) chloride heptahydrate (596 mg, 1.6 mmol) was added at 0° C. The mixture was stirred for 15 min with cooling and then NaBH₄ was added in three portions during 15 min. The reaction mixture was stirred for an additional hour with cooling and then water (10 mL) was added. After stirring for 20 min, the mixture was extracted with EtOAc (4×50 mL). The combined organic solutions were evaporated and the two epimers were separated by repeated silica-gel chromatography using EtOAc as eluent. There was obtained 144 mg (43%) of epimer 1 of (4aR,10aR)-1-propyl-1,2,3,4,4a,5,6,7,8,9,10,10a-dodecahydrobenzo[g]quinolin-6-ol as an off-white powder. ¹H NMR (800 MHZ, CDCl₃): δ 0.86 (t, 3H), 1.02 (qd, 1H), 1.3-1.7 (m, 7H), 1.76 (m, 2H), 1.91 (m, 5H), 2.02 (m, 2H), 2.21 (m, 2H), 2.35 (td, 1H), 2.65 (td, 1H), 2.95 (d, 1H), 4.01 (d, 1H). 13C NMR (201 MHZ, CDCl₃): δ 12.1 (s), 17.5 (s), 19.3 (s), 25.6 (s), 30.5 (s), 32.0 (s), 32.7 (s), 35.1 (s), 36.5 (s), 37.8 (s), 52.9 (s), 55.5 (s), 61.2 (s), 70.0 (s), 129.3 (s), 130.9 (s).

### Example 5

### Epimer 1 of (4aR,10aR)-1-ethyl-1,2,3,4,4a,5,6,7,8,9,10,10a-dodecahydrobenzo[g]quinolin-6-ol

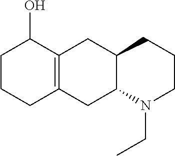 [SMILES: CCN1CCC[C@@H]2CC3=C(CCCC3O)C[C@H]21]

### A) 3-(4-Methoxyphenyl)propanoyl chloride

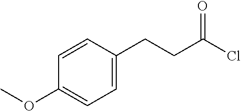

3-(4-Methoxyphenyl) propionic acid (50.0 g, 272 mmol) was dissolved in DCM (500 mL) and to the formed solution was added thionylchloride (49.3 mL, 680 mmol). The mixture was heated to reflux for 6 h, allowed to cool to room temperature and then evaporated to dryness using a rotary evaporator. The crude acid chloride (52.8 g, 98%) was used in the next step without further purification.

### B) N-Ethyl-3-(4-methoxyphenyl) propanamide

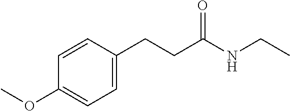

3-(4-Methoxyphenyl)propanoyl chloride (23.9 g, 120 mmol) was dissolved in dry THF (30 mL) and the formed solution was slowly added to an ice-cold mixture of ethylamine (2 M in THF, 150 mL, 300 mmol) and triethylamine (20 mL, 144 mmol) in THF (80 mL). The reaction mixture was stirred at room temperature for one hour and then diluted with aqueous sodium carbonate (10%). The mixture was extracted several times with EtOAc, and the combined organic solutions were washed with brine and dried over sodium sulphate. The solvent was removed by evaporation and there was obtained 19.0 g (76%) of the desired amide which was used in the next step without further purification. GC MS m/z (relative intensity, 70 eV) 208 (10), 207 (71), 135 (23), 134 (79), 122 (9), 121 (bp), 119 (9), 108 (9), 91 (19), 78 (11), 77 (14).

### C) N-Ethyl-3-(4-methoxyphenyl) propan-1-amine

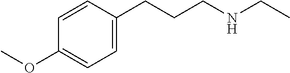

N-ethyl-3-(4-methoxyphenyl) propenamide (19.0 g, 91.7 mmol) was dissolved in dry THF (150 mL) and the formed solution was added dropwise to a mixture of LiAlH₄ (6.96 g, 183 mmol) in THF (120 mL). The reaction mixture was heated to reflux for 3.5 h, cooled with an ice-bath and then diluted with THF (150 mL). After successively quenching with water (7 mL), aqueous NaOH (15%, 7 mL) and finally with water (21 mL), the mixture was stirred for 15 min and then the solids were removed by filtration. The filter cake was washed with EtOH (3×20 mL) and the filtrate was evaporated to dryness using a rotary evaporator. Water (50 mL) was added to the residue and the mixture was extracted several times with EtOAc. The combined organic solutions were washed with brine and then dried over sodium sulphate. The solvent was removed by evaporation and there was obtained 16.8 g (95%) of the desired amine that was used in the next step without further purification. GC MS m/z (relative intensity, 70 eV) 193 (38), 148 (51), 147 (20), 134 (7), 121 (30), 117 (7), 91 (15), 78 (11), 77 (13), 70 (7), 58 (bp).

### D) trans-1-ethyloctahydroquinolin-7 (1H)-one

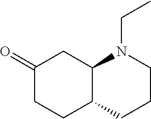

N-Ethyl-3-(4-methoxyphenyl) propylan-1-amine (16.8 g, 87.0 mmol) was dissolved in dry THF (180 mL) in a three necked round bottom flask and the solution was flushed with nitrogen for several minutes before t-butanol (16 mL, 192 mmol) was added. The solution was cooled to −60° C. and then anhydrous ammonia was added via the gas inlet with continues cooling until the volume of the reaction mixture had increased by 180 mL. Metallic lithium (2.35 g, 295 mmol) was slowly added in small portions and the mixture was stirred at −60° C. for 4 h. A mixture of MeOH and saturated aqueous ammonium chloride (1:1, 62 mL) was added to the mixture which then was allowed to warm to room temperature. After carefully heating with a water bath until most of the ammonia had been evaporated from the flask, the pH was adjusted to about 1 by the addition of concentrated hydrochloric acid. The mixture was stirred at room temperature for 18 h and then the pH was adjusted to above 9 at a temperature below 15° C. by the addition of aqueous 4 M NaOH. After extracting the basic mixture several times with DCM, the combined organic solutions were washed with brine and then dried over sodium sulphate. After the solvent was removed by evaporation, the product was purified (and the two stereoisomers were separated) by silica-gel chromatography using EtOAc/MeOH (gradient, 0-10% MeOH) as eluent. There was obtained 4.66 g of the first eluting cis isomer and then 0.51 g of the trans isomer, respectively. The cis isomer was then converted into the desired trans isomer by dissolving 4.66 g in ethanolic KOH (1%, 470 mL), stirring the formed solution at room temperature for four days under a nitrogen atmosphere and the flask covered with aluminium foil. After performing a similar work-up and separation procedures as described above, there was obtained 3.25 g (20%) in total of the racemic trans isomer. GC MS m/z (relative intensity, 70 eV) 181 (14), 166 (7), 125 (10), 124 (bp), 111 (6), 110 (6), 96 (13), 56 (4), 55 (4). ¹H NMR (800 MHZ, CDCl₃) δ 2.94 (dq, 1H), 2.81 (m, 1H), 2.78 (m, 1H), 2.53 (dq, 1H), 2.37 (m, 2H), 2.26 (t, 1H), 2.20 (td, 1H), 2.09 (ddd, 1H), 1.90 (ddt, 1H), 1.81 (dt, 1H), 1.71 (m, 2H), 1.62 (ddd, 1H), 1.36 (tdd, 1H), 1.05 (m, 1H), 0.99 (t, 3H).

### E) (E)-ethyl 4-(trans-1-ethyloctahydroquinolin-7 (1H)-ylidene) butanoate

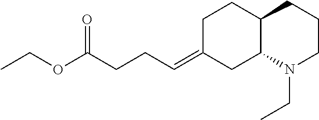 [SMILES: CCOC(=O)CC/C=C1\CC[C@H]2CCCN(CC)[C@@H]2C1]

[3-(Ethoxycarbonyl) propyl]triphenylphosphonium bromide (16.7 g, 35.9 mmol) was dissolved in dry DMF (55 mL) and the formed solution was added dropwise to a cooled (0° C.) solution of potassium tert-butoxide (4.1 g, 35.9 mmol) in DMF (6 mL) under a nitrogen atmosphere. The mixture was stirred with cooling for 30 min. and then a solution of trans-1-ethyloctahydroquinolin-7 (1H)-one (3.25 g, 17.9 mmol) in DMF (7 mL) was added dropwise at 0° C. The reaction mixture was stirred with cooling for 4 h and then for 18 h at room temperature. After cooling with an ice-bath, water (120 mL) was added, and the product was extracted several times with diethyl ether. The organic solutions were successively washed with aqueous LiCl (5%, 75 mL) and brine, dried over sodium sulphate and concentrated to dryness on a rotary evaporator. The residue, which consisted of a mixture of the desired ethyl ester and the corresponding tert-butyl ester as a by-product, was dissolved in ethanol (50 mL) together with concentrated sulfuric acid (1 mL). The mixture was heated to reflux for 18 h and then allowed to cool to room temperature. The solvent was removed by evaporation and the residue diluted with water. The pH was adjusted to over 10 by the addition of saturated aqueous Na₂CO₃ and then the mixture was extracted several times with EtOAc. The combined organic solutions were washed with brine, dried over sodium sulphate, and then evaporated. The product was purified by silica-gel chromatography using isooctane/EtOAc/MeOH (gradient, 0-100% EtOAc and then 0-100% MeOH) as eluent. There was obtained 4.68 g (93%) of the desired ethyl ester as an oil. GC MS m/z (relative intensity, 70 eV) 279 (4), 234 (5), 125 (10), 124 (bp), 111 (2), 110 (2), 96 (5), 91 (2), 79 (2).

### F) (4aR,10aR)-1-ethyl-1,2,3,4,4a,5,8,9,10,10a-decahydrobenzo[g]quinolin-6 (7H)-one

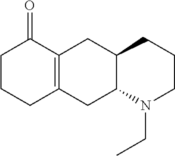 [SMILES: CCN1CCC[C@@H]2CC3=C(CCCC3=O)C[C@H]21]

(E)-ethyl 4-(trans-1-ethyloctahydroquinolin-7 (1H)-ylidene) butanoate (4.67 g, 16.7 mmol) was mixed with Eaton's reagent (18 mL, Phosphorus pentoxide in methanesulfonic acid, 7.7%). The reaction mixture was stirred overnight at 70° C., allowed to cool to room temperature, and then carefully poured into aqueous Na₂CO₃ (10%, 300 mL). After extracting the basic mixture several times with DCM, the combined organic solutions were washed with brine, dried over sodium sulphate, and then concentrated to dryness using a rotary evaporator. The residue was purified by silica-gel chromatography using EtOAc/MeOH (gradient, 0-30% MeOH) as eluent and there was obtained 1.86 g (48%) of the racemic product as an oil. Separating the two enantiomers by repetitive chiral chromatography (Chiralpak® IG, 250×20 mm) using heptane, IPA and DEA (60:40:0.1) as eluent afforded the (+)-enantiomer as the first eluting isomer and the (−)-enantiomer as the last eluting isomer, respectively (approximately 45 mg of the racemic mixture was injected on the column each time). In total, there was obtained 808 mg of the desired (−)-isomer of trans-1-ethyl-1,2,3,4,4a,5,8,9,10,10a-decahydrobenzo[g]quinolin-6 (7H)-one in its non-salt form as an oil consisting of 99.5% of the (−)-isomer and 0.5% of the (+)-isomer as determined by analytical chiral chromatography. Also, there was obtained 795 mg of the (+)-isomer of said compound in its non-salt form as an oil.

Data for the (−)-isomer of trans-1-ethyl-1,2,3,4,4a,5,8,9,10,10a-decahydrobenzo[g]quinolin-6 (7H)-one in its non-salt form: MS(ESI+) m/z 234 [M+H]+. [α]_{D}²⁵=−199° (c=0.05, methanol). ¹H NMR (800 MHZ, CDCl₃) δ 2.95 (m, 1H), 2.87 (m, 1H), 2.53 (m, 3H), 2.42 (m, 1H), 2.36 (m, 1H), 2.26 (m, 2H), 2.18 (m, 2H), 2.05 (td, 1H), 1.97 (m, 2H), 1.85 (m, 1H), 1.68 (m, 3H), 1.41 (m, 1H), 1.05 (m, 4H). The absolute configuration of the two enantiomers was not determined by x-ray crystallography but by comparing the sign of optical rotation as well as elution order on the chiral column with that of the propyl analogue according to Example 4a. Thus, it was concluded that the (−)-isomer has the (4aR,10aR)-configuration and the (+)-isomer has the (4aS,10aS)-configuration.

### G) Epimer 1 of (4aR,10aR)-1-ethyl-1,2,3,4,4a,5,6,7,8,9,10,10a-dodecahydrobenzo[g]quinolin-6-ol

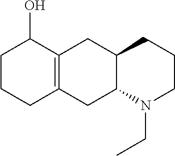 [SMILES: CCN1CCC[C@@H]2CC3=C(CCCC3O)C[C@H]21]

(4aR,10aR)-1-ethyl-1,2,3,4,4a,5,8,9,10,10a-decahydrobenzo[g]quinolin-6 (7H)-one (0.68 g, 2.9 mmol) was dissolved in MeOH (20 mL) and to the formed solution was added cerium (III) chloride heptahydrate (1.3 g, 3.5 mmol) at 0° C. The mixture was stirred with cooling for 15 min. Sodium borohydride (0.33 g, 8.8 mmol) was added in three portions over a period of 15 min and the reaction mixture was stirred for 1 h. Water (30 mL) was added and then stirring was continued for 20 min. After extracting the mixture seven times with EtOAc, the combined organic solutions were washed with brine, dried over sodium sulphate, and then concentrated to dryness using a rotary evaporator. The residue was purified by silica-gel chromatography using EtOAc/MeOH (gradient, 0-50% MeOH) as eluent and there was obtained 0.14 g (16%) of the first eluting isomer as an oil. Also, there was obtained 43 mg of epimer 2 as an oil. Data for epimer 1 of (4aR,10aR)-1-ethyl-1,2,3,4,4a,5,6,7,8,9,10,10a-dodecahydrobenzo[g]quinolin-6-ol: MS(ESI+) m/z 236 [M+H]+. ¹H NMR (800 MHZ, CDCl₃) δ 4.02 (q, 1H), 2.92 (m, 1H), 2.84 (m, 1H), 2.55 (m, 1H), 2.22 (m, 2H), 1.5-2.1 (m, 14H), 1.04 (m, 1H), 1.00 (m, 3H).

### Example 6

### Epimer 1 of (4aR,10aR)-1-methyl-1,2,3,4,4a,5,6,7,8,9,10,10a-dodecahydrobenzo[g]quinolin-6-ol

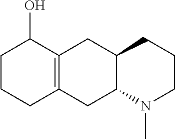 [SMILES: CN1CCC[C@@H]2CC3=C(CCCC3O)C[C@H]21]

### A) trans-1-methyloctahydroquinolin-6 (2H)-one

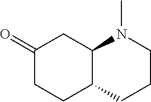

trans-1-methyloctahydroquinolin-6 (2H)-one was synthesized in a similar fashion as in 5B-5D but using methylamine rather than ethylamine as starting material. Starting with 22 g (111 mmol) of 3-(4-methoxyphenyl)propanoyl chloride and 150 mL of methylamine in THF (2M, 300 mmol) afforded 1 g (6%) of trans-1-methyloctahydroquinolin-6 (2H)-one as an oil. GC MS m/z (relative intensity, 70 eV) 168 (2), 167 (14), 166 (5), 124 (3), 111 (10), 110 (bp), 108 (2), 97 (10), 96 (11), 95 (2), 94 (2), 82 (5), 81 (2), 70 (4), 68 (3), 67 (3), 55 (4), 54 (3), 53 (2). ¹H NMR (800 MHZ, CDCl₃) δ 2.90 (m, 1H), 2.81 (m, 1H), 2.2-2.4 (m, 4H), 2.06 (m, 1H), 1.90 (m, 1H), 1.7-1.8 (m, 4H), 1.60 (m, 1H), 1.35 (tdd, 1H), 1.06 (m, 1H).

### B) (E)-ethyl 4-(trans-1-methyloctahydroquinolin-7 (1H)-ylidene) butanoate

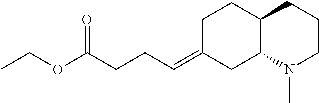 [SMILES: CCOC(=O)CC/C=C1\CC[C@H]2CCCN(C)[C@@H]2C1]

(E)-ethyl 4-(trans-1-methyloctahydroquinolin-7 (1H)-ylidene) butanoate was synthesized in a similar fashion as in 5E but trans-1-methyloctahydroquinolin-6 (2H)-one rather than trans-1-ethyloctahydroquinolin-6 (2H)-one as starting material. Starting with 2.3 g (14 mmol) of (E)-ethyl 4-(trans-1-methyloctahydroquinolin-7 (1H)-ylidene) butanoate afforded 3.6 g (100%) of (E)-ethyl 4-(trans-1-methyloctahydroquinolin-7 (1H)-ylidene) butanoate as an oil.

### C) (4aR,10aR)-1-methyl-1,2,3,4,4a,5,8,9,10,10a-decahydrobenzo[g]quinolin-6 (7H)-one

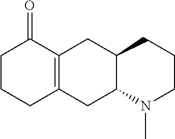 [SMILES: CN1CCC[C@@H]2CC3=C(CCCC3=O)C[C@H]21]

The (−)-isomer of trans-1-methyl-1,2,3,4,4a,5,8,9,10,10a-decahydrobenzo[g]quinolin-6 (7H)-one was synthesized in a similar fashion as in 5F but using (E)-ethyl 4-(trans-1-methyloctahydroquinolin-7 (1H)-ylidene) butanoate rather than (E)-ethyl 4-(trans-1-ethyloctahydroquinolin-7 (1H)-ylidene) butanoate. Starting with 3.2 g (12 mmol) of (E)-ethyl 4-(trans-1-methyloctahydroquinolin-7 (1H)-ylidene) butanoate afforded 0.53 g (20%) of the (−)-isomer of trans-1-methyl-1,2,3,4,4a,5,8,9,10,10a-decahydrobenzo[g]quinolin-6 (7H)-one as an oil.

MS(ESI+) m/z 220 [M+H]+. [α]_{D}²⁵=−244° (c=0.05, methanol). ¹H NMR (800 MHZ, CDCl₃) δ 2.88 (m, 1H), 2.53 (m, 3H), 2.42 (m, 1H), 2.36 (m, 1H), 2.27 (m, 2H), 2.14 (m, 2H), 1.97 (m, 2H), 1.85 (m, 1H), 1.74 (m, 1H), 1.6-1.7 (m, 3H), 1.40 (m, 1H), 1.06 (m, 1H). The absolute configuration of the two enantiomers was not determined by x-ray crystallography but by comparing the sign of optical rotation as well as elution order on the chiral column with that of the propyl analogue according to Example 4a. Thus, it was concluded that the (−)-isomer has the (4aR,10aR)-configuration and the (+)-isomer has the (4aS,10aS)-configuration.

### D) Epimer 1 of (4aR,10aR)-1-methyl-1,2,3,4,4a,5,6,7,8,9,10,10a-dodecahydrobenzo[g]quinolin-6-ol

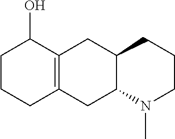 [SMILES: CN1CCC[C@@H]2CC3=C(CCCC3O)C[C@H]21]

Epimer 1 of (4aR,10aR)-1-methyl-1,2,3,4,4a,5,6,7,8,9,10,10a-dodecahydrobenzo[g]quinolin-6-ol was synthesized in a similar fashion as in 5G but using (4aR,10aR)-1-methyl-1,2,3,4,4a,5,8,9,10,10a-decahydrobenzo[g]quinolin-6 (7H)-one rather than (4aR,10aR)-1-ethyl-1,2,3,4,4a,5,8,9,10,10a-decahydrobenzo[g]quinolin-6 (7H)-one. Starting with 460 mg (2 mmol) of (4aR,10aR)-1-methyl-1,2,3,4,4a,5,8,9,10,10a-decahydrobenzo[g]quinolin-6 (7H)-one afforded 0.136 mg (29%) of the epimer 1 of (4aR,10aR)-1-methyl-1,2,3,4,4a,5,6,7,8,9,10,10a-dodecahydrobenzo[g]quinolin-6-ol as an oil.

MS (ESI+) m/z 222 [M+H]+. ¹H NMR (800 MHZ, CDCl₃) δ 4.01 (t, 1H), 2.25 (s, 3H), 2.23 (m, 1H), 2.09 (td, 1H), 2.00 (m, 1H), 1.95 (m, 1H), 1.90 (m, 4H), 1.4-1.8 (m, 10H), 1.04 (m, 1H).

### Example 7

### D-tartaric acid salt of (4aR,6R,10aR)-1-propyl-1,2,3,4,4a,5,6,7,8,9,10,10a-dodecahydrobenzo[g]quinolin-6-ol

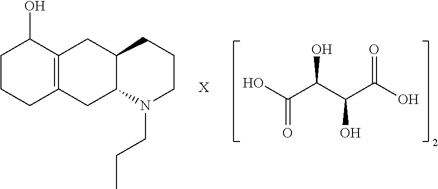

Epimer 1 of (4aR,10aR)-1-propyl-1,2,3,4,4a,5,6,7,8,9,10,10a-dodecahydrobenzo[g]quinolin-6-ol, obtained in a similar fashion as in Example 4B (50 mg, 0.20 mmol), was suspended in EtOAc (2 mL) together with D-tartaric acid (30 mg, 0.20 mmol). The suspension soon became clear and was thereafter stirred at room temperature overnight without any precipitation being formed. The mixture was placed in a fume hood for 28 days. A formed precipitate was isolated by filtration and the solid was washed with EtOAc and then dried under vacuum. There was obtained 56 mg (51%) of the desired D-tartrate salt as a white powder. Melting point: 111.6° C. MS(ESI+) m/z 250 [M+H]+. ¹H NMR (700 MHZ, DMSO-d₆) δ 4.14 (s, 4H), 3.84 (m, 1H), 3.39 (m, 1H), 3.07 (m, 1H), 2.89 (m, 3H), 2.36 (m, 1H), 2.16 (m, 1H), 2.00 (m, 2H), 1.7-1.9 (m, 7H), 1.65 (m, 3H), 1.41 (m, 2H), 1.20 (m, 1H), 0.91 (t, 3H). The NMR spectrum showed a ratio of 1:2 between the above-identified alcohol and D-tartaric acid. Thus, in this example the D-tartaric acid salt of epimer 1 of (4aR,10aR)-1-propyl-1,2,3,4,4a,5,6,7,8,9,10,10a-dodecahydrobenzo[g]quinoline-6-ol is provided as a combination of epimer 1 of (4aR,10aR)-1-propyl-1,2,3,4,4a,5,6,7,8,9,10,10a-dodecahydrobenzo[g]quinolin-6-ol and D-tartaric acid taken in a ratio of 1:2.

### Example 8

Biological activity, Behavior:

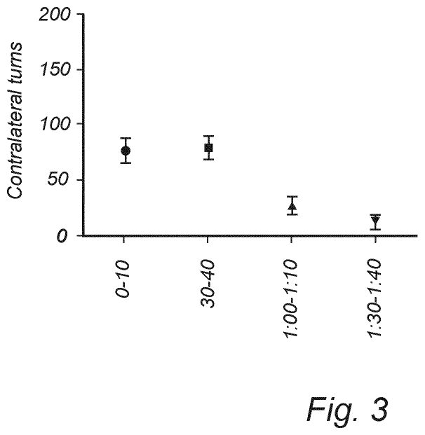

Male Sprague-Dawley rats (150 g; Scanbur, Sweden) were used for this study and housed in separate air-conditioned rooms (12-h dark/light cycle) at 20° C. and a humidity of 53%. Experiments were performed in agreement with the European Communities Council Directive of 24 Nov. 1986 (86/609/EEC) on the ethical use of animals and were approved by the local ethical committee at Karolinska Institute. In most of the experiments, unilaterally 6-OHDA lesioned rats were used to mimic aspects of Parkinsonism. Unilateral 6-OHDA lesioning of nigral dopaminergic axons was performed as previously described (Zhang et al., Neuropharmacology 54 (2008) 1143-1152). Briefly, rats were anesthetized with ketamine (100 mg/kg, i.p.; Parke-Davis, Boxmeer, Netherlands)/xylazine (5 mg/kg, i.p.; Bayer, Kiel, Germany), pretreated with desipramine (25 mg/kg, i.p.; Sigma, Stockholm, Sweden) and pargyline (5 mg/kg, i.p.; Sigma), placed in a stereotaxic instrument and injected with 6-OHDA (2.5 p.I of a 5 mg/ml solution; Sigma) into the medial forebrain bundle of the right hemisphere (AP—2.8 mm, ML—2.0 mm and V—9.0 mm). Two weeks after unilateral 6-OHDA lesioning, rats were administered with apomorphine (1 mg/kg, i.p; Sigma), and their contralateral rotations were measured. The results are shown in FIG. 3. In FIG. 3 the x-axis shows the time in minutes and hours. Only rats which rotated contralaterally more than 100 times were included in further experiments.

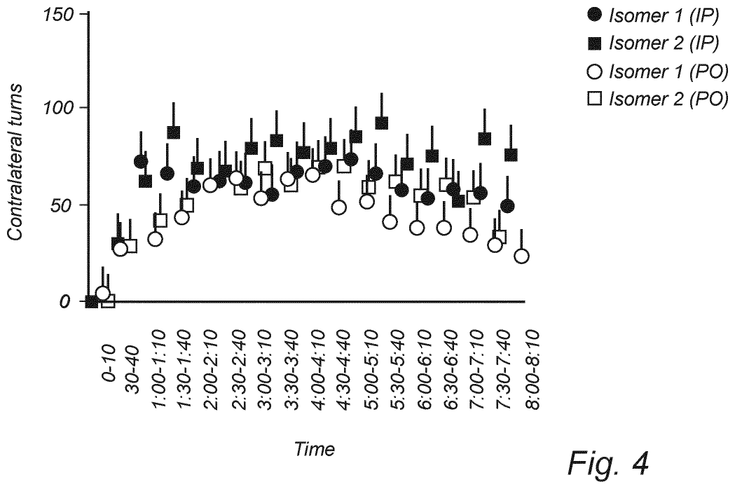

FIG. 4 shows the means for n=4 (IP) and n=6 (PO), respectively, after administration of 0.1 mg/kg of the epimer 1 or epimer 2 of (4aR,10aR)-1-propyl-1,2,3,4,4a,5,6,7,8,9,10,10a-dodecahydrobenzo[g]quinolin-6-ol (one of which is the 6S epimer and the other is the 6R epimer). The time scale is minutes or hours after administration. Data show that both epimers are highly potent dopamine-receptor agonists, with the epimer 2 (the epimer eluted last) being the more potent one. In contrast, the effect of apomorphine declines over time as shown in FIG. 3. In FIG. 3 the x-axis shows minutes or hours after administration. Both epimers indicate good bioavailability (see FIG. 4). Both epimers have also very long duration of action as compared to that of apomorphine (as indicated in FIG. 4). Negative control animals were injected with saline. These animals displayed zero rotations.

### Example 9

m-RNA Analysis:

Animals were killed 60 min after the injection of the drugs by decapitation.

The brains were dissected into a left and a right part. The left part was analyzed for gene expression and dissected into 4 different areas:

Limbic system (containing nucleus accumbens, most parts of the olfactory tubercle, ventral pallidum and amygdala), striatum, frontal cortex, and hippocampus.

Total RNA was prepared by RNeasy Plus Universal Tissue Mini Kit (Qiagen).

RNA pellets were dissolved in RNAse-free water and stored at −80° C. The sample concentration was determined spectrophotometrically by a NanoDrop ND-1000.

A two-step reversed transcription was performed by using a SuperScript III kit (Invitrogen). 1 μg of total RNA was reversed transcribed with 5 μl 2×RT Reaction Mix, 1 μl RT Enzyme and the mix volume was adjusted to 10 μl with RNAse-free water. The sample was incubated at 25° C. for 10 min, 50° C. for 30 min and finally 85° C. for 5 min. 1 U of E. coli RNase H was added following incubation at 37° C. for 20 min and 85° C. for 5 minutes. The cDNA solution was diluted 40 times in Tris EDTA buffer solution pH8 (Merck) and stored at −20° C.

Three sequences (arc and two reference genes) were amplified together in a triplex PCR-reaction. For real-time PCR measurements: 5 μl of the cDNA reaction was amplified in a 20 μl reaction mixture containing 10 μl PerfeCTa MultiPlex qPCR SuperMix (Quantabio), 3.5 μl RNAse-free water, 0.15 UM of each primer and 0.1 μM of each probe. Real-time PCR was measured on CFX96 (Bio-Rad) using the following settings for all genes: 3 min pre-incubation at 95° C. followed by 40 cycles of denaturation at 95° C. for 15 s, annealing and elongation at 60° C. for 1 min. Reference genes are HPRT and cyclophilin.

### Taqman Single and Duplex PCR for Analysis of EGR-1 and Npas4

The real-time PCR reaction consisted of 10 μl Sso Advanced Universal Probes Supermix, 1 μl primer/probe, 1 μl reference gene or 1 μl MQ water and 8 μl of cDNA (diluted 40 times from RT-PCR). Real-time PCR reactions were performed in a CFX96 Real-Time PCR Detector (Bio-Rad) with the following cycling conditions: initial denaturation at 95° C. for 2 min followed by 40 cycles of 95° C. for 5 s and 60° C. for 30 s.

All genes of interest were labelled with the fluorophore FAM on the 5′ end and reference genes (HPRT and ppia (also named cyclophilin)) were labelled with HEX. TaqMan primers and probes were synthesized by Bio-rad (Coralville, Iowa, USA) and used according to the manufacturing protocol.

EGR-1 (Early growth response qRnoCEP0022872) was analyzed in duplex with the reference gene HPRT (hypoxanthine phosphoribosyltransferase qRnoCEP0050840). Npas4 (neuronal PAS domain protein4 qRnoCEP0029461) was analyzed in singleplex. The reference gene Ppia (cyclophilin A peptidyl-propyl cis-trans isomerase qRnoCIP0050815) was also analyzed in order to quantify gene expression for genes of interest.

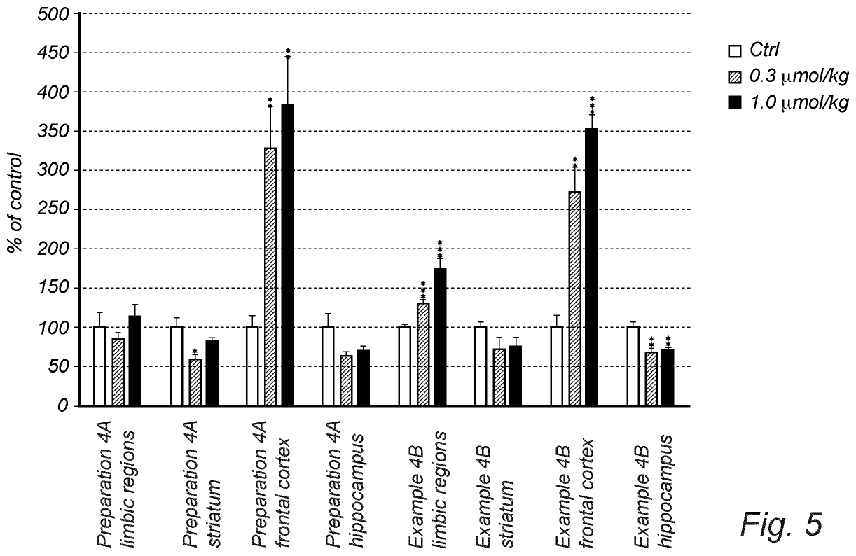

FIG. 5 illustrates the effects on tissue levels of Arc mRNA in four different regions of the brain (limbic regions, striatum, frontal cortex and hippocampus) after subcutaneous administration of two different compounds at two different doses as in comparison to that of corresponding control experiments. The bars in the left half of the figure represent the effects on ARC by the prior art compound according to Preparation 4A ((4aR,10aR)-1-propyl-1,2,3,4,4a,5,8,9,10,10a-decahydrobenzo[g]quinolin-6 (7H)-one) and the bars in the right half of the figure represent the effects on ARC by the compound of the present disclosure according to Example 4B ((4aR,10aR)-1-propyl-1,2,3,4,4a,5,6,7,8,9,10,10a-dodecahydrobenzo[g]quinolin-6-ol). The effects on tissue levels of Arc by both of the two aforementioned compounds were measured at two different doses (0.3 μmol/kg and 1 μmol/kg) and the effect is presented as percent of control means±SEM. Statistical significance was assessed using Student's t-test (2 tailed) vs controls.

As shown in the diagram of FIG. 5, both of the two compounds dose-dependently increase tissue levels of Arc in the frontal cortex, which sometimes is observed for a dopamine receptor agonist. Also shown in the diagram is that the compound according to Example 4B dose-dependently increases the tissue levels of Arc in the limbic regions which is a property that the prior art compound according to Preparation 4A does not have. As Arc is a biomarker of synaptic activity, this attribute of the compound according to Example 4B allows for providing a unique therapeutic profile such as improvements related to emotion, behavior, and/or long-term memory. Furthermore, the compound according to Example 4B increases tissue levels of other genes in the limbic regions such as for instance egr-1 and Npas4. The compound does so to a greater extent as compared to that of the prior art compound according to Preparation 4A and these observed effects allow for an improved therapy for the patients with neurodegenerative diseases and/or neurological disorders.

SEQUENCE LISTING
The primer and probe sequences are as follows for
measuring of arc:
Activity-regulated gene (Arc) (accession number
U19866)
Sense: 5′- GGA GTT CAA GAA GGA GTT TC-3′
(SEQ ID NO: 1)
Antisense: 5′- CCA CAT ACA GTG TCT GGT A -3′
(SEQ ID NO: 2)
Probe: CCG CTT ACG CCA GAG GAA CT
(SEQ ID NO: 3)
Dye: 5′FAM Quencher: 3′BHQ1
Product size: 149
Hypoxantine phosphoribosyl transferase (HPRT)
(accession number AF001282)
Sense: 5′- AGG GAT TTG AAT CAT GTT TG -3′
(SEQ ID NO: 4)
Antisense: 5′- CTG CTA GTT CTT TAC TGG C -3′
(SEQ ID NO: 5)
Probe: TGT AGA TTC AAC TTG CCG CTG TC
SEQ ID NO: 6)
Dye: 5′HEX Quencher: 3′BHQ1
Product size: 121
Cyclophilin A (cyclo)(accession number M19533)
Sense: 5′- CTG GAC CAA ACA CAA ATG-3′
(SEQ ID NO: 7)
Antisense: 5′- ATG CCT TCT TTC ACC TTC -3′
(SEQ ID NO: 8)
Probe: TTG CCA TCC AGC CAC TCA GT
(SEQ ID NO: 9)
Dye: 5′Texas red Quencher: 3′BHQ2
Product size: 100

### Example 10

### Locomotor Activity:

Behavioral activity was measured using eight Digiscan activity monitors (RXYZM (16) TAO, Omnitech Electronics, Columbus, OH, USA), connected to an Omnitech Digiscan analyzer and an Apple Macintosh computer equipped with a digital interface board (NB DIO-24, National Instruments, USA). Each activity monitor consisted of a quadratic metal frame (W×L=40 cm×40 cm) equipped with photo beam sensors. During measurements of behavioral activity, a rat was put in a transparent acrylic cage (W×L×H, 40×40×30 cm) which in turn was placed in the activity monitor. Each activity monitor was equipped with three rows of infrared photo beam sensors, each row consisting of 16 sensors. Two rows were placed along the front and the side of the floor of the cage, at a 90° angle, and the third row was placed 10 cm above the floor to measure vertical activity. Photo beam sensors were spaced 2.5 cm apart. Each activity monitor was fitted in an identical sound and light attenuating box containing a weak house light and a fan.

The computer software was written using object-oriented programming (LabVIEW™ National instruments, Austin, TX, USA).

Behavioral data from each activity monitor, representing the position (horizontal center of gravity and vertical activity) of the animal at each time, were recorded at a sampling frequency of 25 Hz and collected using a custom written LABView™ application. The data from each recording session were stored and analyzed with respect to distance traveled. Each behavioral recording session lasted 180 min, starting approximately 5 min after the injection of test compound.

Compounds disclosed herein have been tested for effects on spontaneous locomotor activity in non-pre-treated Sprague-Dawley rats (based on accumulated distance travelled 0-180 min post dosing), and with the single dose of 0.3 μmol/kg (n=5, SC) compared to a control group of animals (n=5) that obtained saline (SC). The unit of the distance travelled is an arbitrary unit.

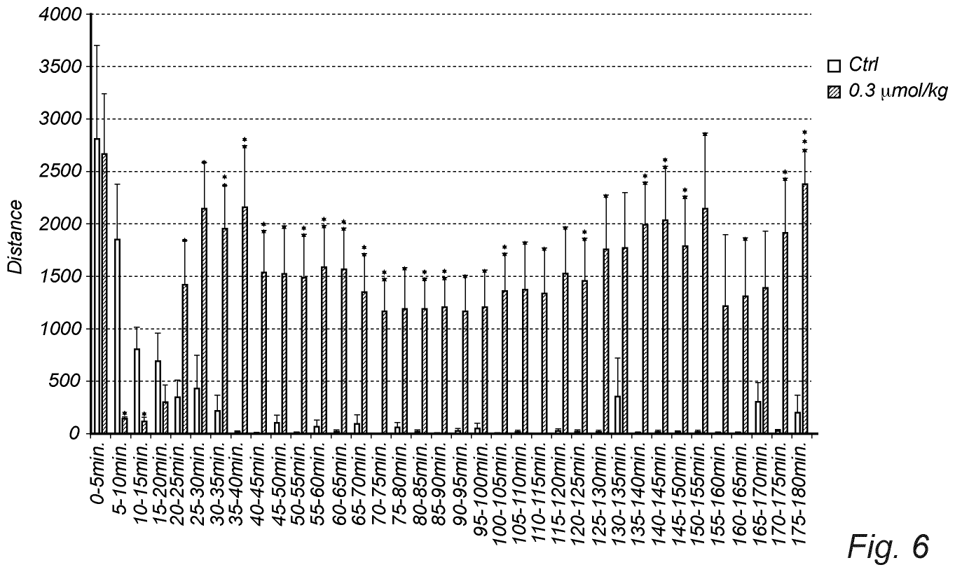

FIG. 6 shows the means of distance travelled after administration of either 0.3 μmol/kg of the prior art compound according to Preparation 4A ((4aR,10aR)-1-propyl-1,2,3,4,4a,5,8,9,10,10a-decahydrobenzo[g]quinolin-6 (7H)-one) or administration of saline (control experiment) to drug-naïve rats. The animals were placed in the motility meters immediately after administration and locomotor activity was recorded for 180 minutes.

Results are presented as distance travelled for the control group (empty bar) and for the group of animals that obtained the drug (filled bar).

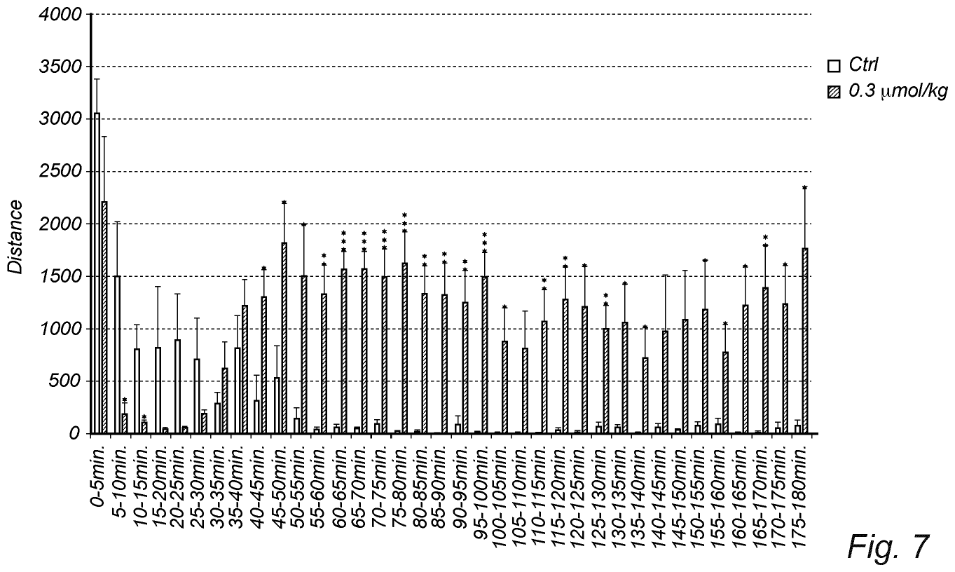

FIG. 7 shows the means of distance travelled after administration of either 0.3 μmol/kg of the compound according to Example 4B (epimer 1 of (4aR,10aR)-1-propyl-1,2,3,4,4a,5,6,7,8,9,10,10a-dodecahydrobenzo[g]quinolin-6-ol) or administration of saline (control experiment) to drug-naïve rats. The animals were placed in the motility meters immediately after administration and locomotor activity was recorded for 180 minutes. Results are presented as distance travelled for the control group (empty bars) and for the group of animals that obtained the drug (filled bar).

As shown in FIG. 6 and FIG. 7, both of the two compounds being tested do affect motor activity patterns in normal, non-pre-treated, rats. Thus, the prior art compound according to Preparation 4A as well as the compound according to Example 4B induce hyperactivity. The desired effect is lasting for at least 180 min for both of the two compounds showing that the two compounds do have a long duration of action. However, a difference between the two compounds is that the on-set of action, i.e. that the distance travelled increases after the initial decrease in distance travelled taking place immediately after administration, for the prior art compound is faster (20-25 min) as compared to the compound according to Example 4B (35-40 min). A further difference is that the immediate effect on motor activity for the prior art compound is steeper with a long distance travelled already within 20-45 min. These data taken together show that the animals obtaining the prior art compound, quickly are having a higher peak plasma concentration of the active species resulting from the administered drug as compared to that of the compound according to Example 4B. Consequently, the data show that the compound according to Example 4B is associated with no or a mild side effect profile due to a slower on-set of action.

### REFERENCES

1. Liu et al., “Extremely Potent Orally Active Benzo[g]quinoline Analogue of the Dopaminergic Prodrug: 6-(N,N-Di-n-propyl)amino-3,4,5,6,7,8-hexahydro-2H-naphtalen-1-one”, J. Med. Chem., 2006, 49, 1494-1498 (The title of this article was soon afterwards corrected to “Extremely Potent Orally Active Benzo[g]quinoline Analogue of the Dopaminergic Prodrug: 1-Propyl-trans-2,3,4,4a,5,7,8,9,10,10a-decahydro-1H-benzo[g]quinoline-6-one”, J. Med. Chem., 2006, 49, 6930)
2. Liu et al., “A novel synthesis and pharmacological evaluation of a potential dopamine D1/D2 agonist: 1-Propyl 1,2,3,4,4a,5,10,10a-octahydrobenzo[g]quinoline-6,7-diol “Bioorganic & Medicinal Chemistry, 16 (2008), 3438-3444
3. WO 2010/097092
4. WO 2001/078713
5. WO 2019/101917
6. Zhang et al., Neuropharmacology 54 (2008) 1143-1152.
7. WO 2020/234270
8. WO 2020/234271
9. WO 2020/234272
10. WO 2020/234273
11. WO 2020/234274
12. WO 2020/234275
13. WO 2020/234276
14. WO 2020/234277

1. A compound of Formula II1: 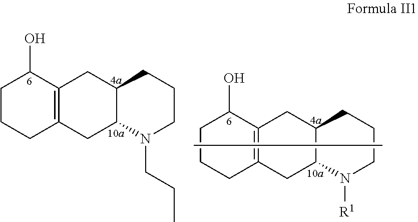
or a pharmaceutically acceptable salt thereof,
wherein
carbon 4a and carbon 10a both have R configuration.

2. The compound according to claim 1, wherein carbon 6 has R configuration thereby providing a compound of Formula IIa1: 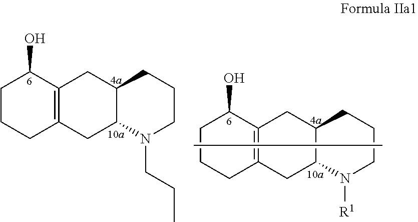
or a pharmaceutically acceptable salt thereof.

3. The compound according to claim 1, wherein carbon 6 has S configuration thereby providing a compound of Formula IIb1: 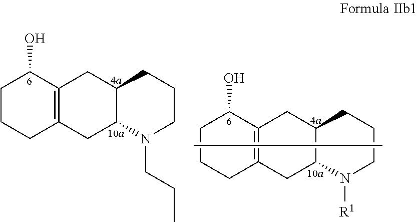
or a pharmaceutically acceptable salt thereof.

4.-7. (canceled)

8. A pharmaceutically acceptable salt of the compound according to claim 1, wherein said pharmaceutically acceptable salt is a combination of said compound of Formula II1 and an organic acid.

9. (canceled)

10. The pharmaceutically acceptable salt according to claim 8, wherein said pharmaceutically acceptable salt is a combination of the compound of Formula II and said organic acid in a ratio of 1:1, 2:1 or 1:2.

11. The pharmaceutically acceptable salt according to claim 10, wherein said salt pharmaceutically acceptable is a combination of the compound of Formula II and said organic acid in a ratio of 1:2.

12. The pharmaceutically acceptable salt according to claim 10, wherein said organic acid is D-tartaric acid.

13. The compound according to claim 1, or a pharmaceutically acceptable salt thereof,
wherein the compound is in solid form.

14. The compound according to claim 1, or a pharmaceutically acceptable salt thereof wherein the compound is in crystalline form.

15. A pharmaceutical composition comprising a therapeutically acceptable amount of a compound according to claim 1, or a pharmaceutically acceptable salt thereof in admixture with at least one pharmaceutically acceptable carrier, excipient and/or diluent.

16.-28. (canceled)

29. A method for treatment of one or more of the following:
Parkinson's disease, Huntington's disease, Restless leg syndrome, Alzheimer's disease, schizophrenia, attention deficit hyperactivity disorder, and drug addiction, said method comprising administering to a mammal in need thereof,
a therapeutically effective amount of:
the compound according to claim 1, or a pharmaceutically acceptable salt thereof.

30. The method according to claim 29, wherein the mammal is a human.

31. The method according to claim 29, wherein the treatment comprises treatment of Parkinson's disease.

32. The method according to claim 29, wherein the treatment is associated with no or mild side effect(s).

33. The method according to claim 32, wherein the mild side effect(s) comprise(s) of nausea and/or vomiting.

34. The method according to claim 31, wherein the treatment further comprises treatment of non-motor symptom(s) associated with Parkinson's disease.

35. The method according to claim 34, wherein the non-motor symptom(s) associated with Parkinson's disease comprise(s) one or more of the following: cognitive functional decline, depression, anxiety, apathy, and Parkinson's disease dementia.

36. A method for preparing the compound of Formula II1 according to claim 1, or a pharmaceutically acceptable salt thereof, said method comprising the steps of:
a) reducing a compound of Formula I 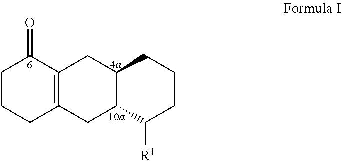
wherein R¹ is n-propyl,
with a reducing agent in the presence of a Lewis acid and a solvent
whereby the carbonyl group is reduced into a hydroxyl group thereby providing the compound of Formula II1, and
b) optionally separating the compound of Formula II1 into a compound of Formula IIa1 and Formula IIb1, and
c) optionally combining the compound of Formula II1 obtained in step a) or step b) with a pharmaceutically acceptable acid thereby providing a pharmaceutically acceptable salt of the compound of Formula II1.

37. (canceled)

38. The pharmaceutically acceptable salt according to claim 8, wherein said pharmaceutically acceptable salt is a combination of the compound of Formula IIa1 and D-tartaric acid in a ration of 1:2.
